SEQUENCE LISTING (50 sequences)

SEQ ID NO: 1 - protein, 534 aa, synthetic construct
  REGION 1..534: note = LAIR2-Fc(IgG1)-TGFb Trap (TGFbRII)
MEWSWVFLFFLSVTTGVHSQEGALPRPSISAEPGTVISPGSHVTFMCRGPVGVQTFRLEREDRAKYKDSYNVFRLGPSESEARFHIDSVSEGNAGLYRCLYYKPPGWSEHSDFLELLVKESSGGPDSPDTEPGSSAGTVPGTEASGFDAPDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSGGGGSGGGGSGGGGSGIPPHVQKSVNNDMIVTDNNGAVKFPQLCKFCDVRFSTCDNQKSCMSNCSITSICEKPQEVCVAVWRKNDENITLETVCHDPKLPYHDFILEDAASPKCIMKEKKKPGETFFMCSCSSDECNDNIIFSEEYNTSNPD

SEQ ID NO: 2 - protein, 900 aa, synthetic construct
  REGION 1..900: note = LAIR2-Fc(IgG1)-IL12/LP-single chain IL12-linker-LAIR2(21-152)-Fc(IgG1)
MEWSWVFLFFLSVTTGVHSIWELKKDVYVVELDWYPDAPGEMVVLTCDTPEEDGITWTLDQSSEVLGSGKTLTIQVKEFGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLKYENYTSSFFIRDIIKPDPPKNLQLKPLKNSRQVEVSWEYPDTWSTPHSYFSLTFSVQVQGKSKREKKDRVFTDKTSATVICRKNASISVRAQDRYYSSSWSEWASVPCSGGGGSGGGGSGGGGSRNLPVATPDPGMFPCLHHSQNLLRAVSNMLQKARQTLEFYPCTSEEIDHEDITKDKTSTVEACLPLELTKNESCLNSRETSFITNGSCLASRKTSFMMALCLSSIYEDLKMYQVEFKTMNAKLLMDPKRQIFLDQNMLAVIDELMQALNFNSETVPQKSSLEEPDFYKTKIKLCILLHAFRIRAVTIDRVMSYLNASGGSGGQEGALPRPSISAEPGTVISPGSHVTFMCRGPVGVQTFRLEREDRAKYKDSYNVFRLGPSESEARFHIDSVSEGNAGLYRCLYYKPPGWSEHSDFLELLVKESSGGPDSPDTEPGSSAGTVPGTEASGFDAPDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 3 - protein, 233 aa, synthetic construct
  REGION 1..233: note = TNT-TGF-Beta-trap (N830, Light Chain Orf including leader peptide (LP-Hu51VL-C-Kappa)
MEWSWVFLFFLSVTTGVHSEIVLTQSPATLSLSPGERATLSCRARQSISNYLHWYQQKPGQAPRLLIYYASQSISGIPDRFSGSGSGTDFTLTISRLEPEDFAVYYCQQSNSWPLTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 4 - protein, 627 aa, synthetic construct
  REGION 1..627: note = TNT-TGF-Beta-trap (N830), Heavy Chain Orf including leader peptide (LP-Hu51VH-CH(IgG1)-linker-TGF-Beta-RII
MEWSWVFLFFLSVTTGVHSQVQLVQSGAEVKKPGASVKVSCKASGYTFTRYWMHWVRQAPGQGLEWIGAIYPGNSDTSYNQKFKGKATITADTSTNTAYMELSSLRSEDTAVYYCARGEEIGSRRWFAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSGGGGSGGGGSGGGGSGIPPHVQKSVNNDMIVTDNNGAVKFPQLCKFCDVRFSTCDNQKSCMSNCSITSICEKPQEVCVAVWRKNDENITLETVCHDPKLPYHDFILEDAASPKCIMKEKKKPGETFFMCSCSSDECNDNIIFSEEYNTSNPD

SEQ ID NO: 5 - protein, 358 aa, synthetic construct
  REGION 1..358: note = LAIR2/IgG1Fc
QEGALPRPSISAEPGTVISPGSHVTFMCRGPVGVQTFRLEREDRAKYKDSYNVFRLGPSESEARFHIDSVSEGNAGLYRCLYYKPPGWSEHSDFLELLVKESSGGPDSPDTEPGSSAGTVPGTEASGFDAPDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 6 - protein, 358 aa, synthetic construct
  REGION 1..358: note = LAIR2/IgG4Fc-PE
QEGALPRPSISAEPGTVISPGSHVTFMCRGPVGVQTFRLEREDRAKYKDSYNVFRLGPSESEARFHIDSVSEGNAGLYRCLYYKPPGWSEHSDFLELLVKESSGGPDSPDTEPGSSAGTVPGTEASGFDAPKYGPPCPPCPAPEFEGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 7 - protein, 364 aa, synthetic construct
  REGION 1..364: note = LAIR2/mIgG2a
QEGALPRPSISAEPGTVISPGSHVTFMCRGPVGVQTFRLEREDRAKYKDSYNVFRLGPSESEARFHIDSVSEGNAGLYRCLYYKPPGWSEHSDFLELLVKESSGGPDSPDTEPGSSAGTVPGTEASGFDAPEPRGPTIKPCPPCKCPAPNLLGGPSVFIFPPKIKDVLMISLSPIVTCVVVDVSEDDPDVQISWFVNNVEVHTAQTQTHREDYNSTLRVVSALPIQHQDWMSGKEFKCKVNNKDLPAPIERTISKPKGSVRAPQVYVLPPPEEEMTKKQVTLTCMVTDFMPEDIYVEWTNNGKTELNYKNTEPVLDSDGSYFMYSKLRVEKKNWVERNSYSCSVVHEGLHNHHTTKSFSRTPGK

SEQ ID NO: 8 - protein, 894 aa, synthetic construct
  REGION 1..894: note = LAIR2/Fc/IL-12PR (Protease Resistant)
QEGALPRPSISAEPGTVISPGSHVTFMCRGPVGVQTFRLEREDRAKYKDSYNVFRLGPSESEARFHIDSVSEGNAGLYRCLYYKPPGWSEHSDFLELLVKESSGGPDSPDTEPGSSAGTVPGTEASGFDAPDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSGGGGSGGGGSGGGGSIWELKKDVYVVELDWYPDAPGEMVVLTCDTPEEDGITWTLDQSSEVLGSGKTLTIQVKEFGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLKYENYTSSFFIRDIIKPDPPKNLQLKPLKNSRQVEVSWEYPDTWSTPHSYFSLTFSVQVQGKDNTEGRVFTDKTSATVICRKNASISVRAQDRYYSSSWSEWASVPCSGGGGSGGGGSGGGGSRNLPVATPDPGMFPCLHHSQNLLRAVSNMLQKARQTLEFYPCTSEEIDHEDITKDKTSTVEACLPLELTKNESCLNSRETSFITNGSCLASRKTSFMMALCLSSIYEDLKMYQVEFKTMNAKLLMDPKRQIFLDQNMLAVIDELMQALNFNSETVPQKSSLEEPDFYKTKIKLCILLHAFRIRAVTIDRVMSYLNAS

SEQ ID NO: 9 - protein, 894 aa, synthetic construct
  REGION 1..894: note = LAIR2/Fc(S254A)/IL-12PR (Weaker FcRN binding. Faster clearance)
QEGALPRPSISAEPGTVISPGSHVTFMCRGPVGVQTFRLEREDRAKYKDSYNVFRLGPSESEARFHIDSVSEGNAGLYRCLYYKPPGWSEHSDFLELLVKESSGGPDSPDTEPGSSAGTVPGTEASGFDAPDKTHTCPPCPAPELLGGPSVFLFPPKPKDTLMIARTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSGGGGSGGGGSGGGGSIWELKKDVYVVELDWYPDAPGEMVVLTCDTPEEDGITWTLDQSSEVLGSGKTLTIQVKEFGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLKYENYTSSFFIRDIIKPDPPKNLQLKPLKNSRQVEVSWEYPDTWSTPHSYFSLTFSVQVQGKDNTEGRVFTDKTSATVICRKNASISVRAQDRYYSSSWSEWASVPCSGGGGSGGGGSGGGGSRNLPVATPDPGMFPCLHHSQNLLRAVSNMLQKARQTLEFYPCTSEEIDHEDITKDKTSTVEACLPLELTKNESCLNSRETSFITNGSCLASRKTSFMMALCLSSIYEDLKMYQVEFKTMNAKLLMDPKRQIFLDQNMLAVIDELMQALNFNSETVPQKSSLEEPDFYKTKIKLCILLHAFRIRAVTIDRVMSYLNAS

SEQ ID NO: 10 - protein, 894 aa, synthetic construct
  REGION 1..894: note = LAIR2/Fc(H435A)/IL-12PR (Weaker FcRN binding. Faster clearance)
QEGALPRPSISAEPGTVISPGSHVTFMCRGPVGVQTFRLEREDRAKYKDSYNVFRLGPSESEARFHIDSVSEGNAGLYRCLYYKPPGWSEHSDFLELLVKESSGGPDSPDTEPGSSAGTVPGTEASGFDAPDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNAYTQKSLSLSPGKGGGGSGGGGSGGGGSGGGGSIWELKKDVYVVELDWYPDAPGEMVVLTCDTPEEDGITWTLDQSSEVLGSGKTLTIQVKEFGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLKYENYTSSFFIRDIIKPDPPKNLQLKPLKNSRQVEVSWEYPDTWSTPHSYFSLTFSVQVQGKDNTEGRVFTDKTSATVICRKNASISVRAQDRYYSSSWSEWASVPCSGGGGSGGGGSGGGGSRNLPVATPDPGMFPCLHHSQNLLRAVSNMLQKARQTLEFYPCTSEEIDHEDITKDKTSTVEACLPLELTKNESCLNSRETSFITNGSCLASRKTSFMMALCLSSIYEDLKMYQVEFKTMNAKLLMDPKRQIFLDQNMLAVIDELMQALNFNSETVPQKSSLEEPDFYKTKIKLCILLHAFRIRAVTIDRVMSYLNAS

SEQ ID NO: 11 - protein, 894 aa, synthetic construct
  REGION 1..894: note = LAIR2/Fc(S254A/H435A)/IL-12PR (Weaker FcRN binding. Faster clearance)
QEGALPRPSISAEPGTVISPGSHVTFMCRGPVGVQTFRLEREDRAKYKDSYNVFRLGPSESEARFHIDSVSEGNAGLYRCLYYKPPGWSEHSDFLELLVKESSGGPDSPDTEPGSSAGTVPGTEASGFDAPDKTHTCPPCPAPELLGGPSVFLFPPKPKDTLMIARTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNAYTQKSLSLSPGKGGGGSGGGGSGGGGSGGGGSIWELKKDVYVVELDWYPDAPGEMVVLTCDTPEEDGITWTLDQSSEVLGSGKTLTIQVKEFGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLKYENYTSSFFIRDIIKPDPPKNLQLKPLKNSRQVEVSWEYPDTWSTPHSYFSLTFSVQVQGKDNTEGRVFTDKTSATVICRKNASISVRAQDRYYSSSWSEWASVPCSGGGGSGGGGSGGGGSRNLPVATPDPGMFPCLHHSQNLLRAVSNMLQKARQTLEFYPCTSEEIDHEDITKDKTSTVEACLPLELTKNESCLNSRETSFITNGSCLASRKTSFMMALCLSSIYEDLKMYQVEFKTMNAKLLMDPKRQIFLDQNMLAVIDELMQALNFNSETVPQKSSLEEPDFYKTKIKLCILLHAFRIRAVTIDRVMSYLNAS

SEQ ID NO: 12 - protein, 233 aa, synthetic construct; identical to SEQ ID NO: 3
  REGION 1..233: note = TNT-IL-12 (N812) Light Chain Orf including leader peptide (LP-Hu51VL-Ck)

SEQ ID NO: 13 - protein, 990 aa, synthetic construct
  REGION 1..990: note = TNT-IL-12 (N812) Heavy Chain Orf including leader peptide (LP-IL12b(p40)(C272S)-linker-IL12a(p35)-Hu51VH-CH(IgG1))
MEWSWVFLFFLSVTTGVHSIWELKKDVYVVELDWYPDAPGEMVVLTCDTPEEDGITWTLDQSSEVLGSGKTLTIQVKEFGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLKYENYTSSFFIRDIIKPDPPKNLQLKPLKNSRQVEVSWEYPDTWSTPHSYFSLTFSVQVQGKSKREKKDRVFTDKTSATVICRKNASISVRAQDRYYSSSWSEWASVPCSGGGGSGGGGSGGGGSRNLPVATPDPGMFPCLHHSQNLLRAVSNMLQKARQTLEFYPCTSEEIDHEDITKDKTSTVEACLPLELTKNESCLNSRETSFITNGSCLASRKTSFMMALCLSSIYEDLKMYQVEFKTMNAKLLMDPKRQIFLDQNMLAVIDELMQALNFNSETVPQKSSLEEPDFYKTKIKLCILLHAFRIRAVTIDRVMSYLNASLEQVQLVQSGAEVKKPGASVKVSCKASGYTFTRYWMHWVRQAPGQGLEWIGAIYPGNSDTSYNQKFKGKATITADTSTNTAYMELSSLRSEDTAVYYCARGEEIGSRRWFAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 14 - protein, 19 aa, synthetic construct
  REGION 1..19: note = Leader peptide
MEWSWVFLFFLSVTTGVHS

SEQ ID NO: 15 - protein, 131 aa, synthetic construct
  REGION 1..131: note = LAIR2
QEGALPRPSISAEPGTVISPGSHVTFMCRGPVGVQTFRLEREDRAKYKDSYNVFRLGPSESEARFHIDSVSEGNAGLYRCLYYKPPGWSEHSDFLELLVKESSGGPDSPDTEPGSSAGTVPGTEASGFDAP

SEQ ID NO: 16 - protein, 227 aa, synthetic construct
  REGION 1..227: note = Fc(IgG1)
DKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 17 - protein, 20 aa, synthetic construct
  REGION 1..20: note = Linker
GGGGSGGGGSGGGGSGGGGS

SEQ ID NO: 18 - protein, 137 aa, synthetic construct
  REGION 1..137: note = TGFbRII
GIPPHVQKSVNNDMIVTDNNGAVKFPQLCKFCDVRFSTCDNQKSCMSNCSITSICEKPQEVCVAVWRKNDENITLETVCHDPKLPYHDFILEDAASPKCIMKEKKKPGETFFMCSCSSDECNDNIIFSEEYNTSNPD

SEQ ID NO: 19 - protein, 518 aa, synthetic construct
  REGION 1..518: note = Single chain IL12: IL12b(p40)(C272S)-linker-IL12a(p35)
IWELKKDVYVVELDWYPDAPGEMVVLTCDTPEEDGITWTLDQSSEVLGSGKTLTIQVKEFGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLKYENYTSSFFIRDIIKPDPPKNLQLKPLKNSRQVEVSWEYPDTWSTPHSYFSLTFSVQVQGKSKREKKDRVFTDKTSATVICRKNASISVRAQDRYYSSSWSEWASVPCSGGGGSGGGGSGGGGSRNLPVATPDPGMFPCLHHSQNLLRAVSNMLQKARQTLEFYPCTSEEIDHEDITKDKTSTVEACLPLELTKNESCLNSRETSFITNGSCLASRKTSFMMALCLSSIYEDLKMYQVEFKTMNAKLLMDPKRQIFLDQNMLAVIDELMQALNFNSETVPQKSSLEEPDFYKTKIKLCILLHAFRIRAVTIDRVMSYLNAS

SEQ ID NO: 20 - protein, 306 aa, synthetic construct
  REGION 1..306: note = IL-12b(p40)(C272S)
IWELKKDVYVVELDWYPDAPGEMVVLTCDTPEEDGITWTLDQSSEVLGSGKTLTIQVKEFGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLKYENYTSSFFIRDIIKPDPPKNLQLKPLKNSRQVEVSWEYPDTWSTPHSYFSLTFSVQVQGKSKREKKDRVFTDKTSATVICRKNASISVRAQDRYYSSSWSEWASVPCS

SEQ ID NO: 21 - protein, 15 aa, synthetic construct
  REGION 1..15: note = Linker
GGGGSGGGGSGGGGS

SEQ ID NO: 22 - protein, 197 aa, synthetic construct
  REGION 1..197: note = IL12a(p35)
RNLPVATPDPGMFPCLHHSQNLLRAVSNMLQKARQTLEFYPCTSEEIDHEDITKDKTSTVEACLPLELTKNESCLNSRETSFITNGSCLASRKTSFMMALCLSSIYEDLKMYQVEFKTMNAKLLMDPKRQIFLDQNMLAVIDELMQALNFNSETVPQKSSLEEPDFYKTKIKLCILLHAFRIRAVTIDRVMSYLNAS

SEQ ID NO: 23 - protein, 5 aa, synthetic construct
  REGION 1..5: note = Linker
GGSGG

SEQ ID NO: 24 - protein, 107 aa, synthetic construct
  REGION 1..107: note = Hu51VL
EIVLTQSPATLSLSPGERATLSCRARQSISNYLHWYQQKPGQAPRLLIYYASQSISGIPDRFSGSGSGTDFTLTISRLEPEDFAVYYCQQSNSWPLTFGQGTKVEIK

SEQ ID NO: 25 - protein, 107 aa, synthetic construct [sequence omitted]

SEQ ID NO: 26 - protein, 121 aa, synthetic construct
  REGION 1..121: note = Hu51VH
QVQLVQSGAEVKKPGASVKVSCKASGYTFTRYWMHWVRQAPGQGLEWIGAIYPGNSDTSYNQKFKGKATITADTSTNTAYMELSSLRSEDTAVYYCARGEEIGSRRWFAYWGQGTLVTVSS

SEQ ID NO: 27 - protein, 330 aa, synthetic construct [sequence omitted]

SEQ ID NO: 28 - protein, 137 aa, synthetic construct; identical to SEQ ID NO: 18
  REGION 1..137: note = TGFbRII

SEQ ID NO: 29 - protein, 881 aa, synthetic construct
  REGION 1..881: note = IL-12/LAIR2/IgG1Fc
IWELKKDVYVVELDWYPDAPGEMVVLTCDTPEEDGITWTLDQSSEVLGSGKTLTIQVKEFGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLKYENYTSSFFIRDIIKPDPPKNLQLKPLKNSRQVEVSWEYPDTWSTPHSYFSLTFSVQVQGKSKREKKDRVFTDKTSATVICRKNASISVRAQDRYYSSSWSEWASVPCSGGGGSGGGGSGGGGSRNLPVATPDPGMFPCLHHSQNLLRAVSNMLQKARQTLEFYPCTSEEIDHEDITKDKTSTVEACLPLELTKNESCLNSRETSFITNGSCLASRKTSFMMALCLSSIYEDLKMYQVEFKTMNAKLLMDPKRQIFLDQNMLAVIDELMQALNFNSETVPQKSSLEEPDFYKTKIKLCILLHAFRIRAVTIDRVMSYLNASGGSGGQEGALPRPSISAEPGTVISPGSHVTFMCRGPVGVQTFRLEREDRAKYKDSYNVFRLGPSESEARFHIDSVSEGNAGLYRCLYYKPPGWSEHSDFLELLVKESSGGPDSPDTEPGSSAGTVPGTEASGFDAPDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 30 - protein, 879 aa, synthetic construct
  REGION 1..879: note = IL-12/LAIR2/IgG1Fc
IWELKKDVYVVELDWYPDAPGEMVVLTCDTPEEDGITWTLDQSSEVLGSGKTLTIQVKEFGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLKYENYTSSFFIRDIIKPDPPKNLQLKPLKNSRQVEVSWEYPDTWSTPHSYFSLTFSVQVQGKDNTEGRVFTDKTSATVICRKNASISVRAQDRYYSSSWSEWASVPCSGGGGSGGGGSGGGGSRNLPVATPDPGMFPCLHHSQNLLRAVSNMLQKARQTLEFYPCTSEEIDHEDITKDKTSTVEACLPLELTKNESCLNSRETSFITNGSCLASRKTSFMMALCLSSIYEDLKMYQVEFKTMNAKLLMDPKRQIFLDQNMLAVIDELMQALNFNSETVPQKSSLEEPDFYKTKIKLCILLHAFRIRAVTIDRVMSYLNASGGSGGQEGALPRPSISAEPGTVISPGSHVTFMCRGPVGVQTFRLEREDRAKYKDSYNVFRLGPSESEARFHIDSVSEGNAGLYRCLYYKPPGWSEHSDFLELLVKESSGGPDSPDTEPGSSAGTVPGTEASGFDAPDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 31 - protein, 515 aa, synthetic construct
  REGION 1..515: note = LAIR-2/IgG1Fc/TGF-Beta-RII
QEGALPRPSISAEPGTVISPGSHVTFMCRGPVGVQTFRLEREDRAKYKDSYNVFRLGPSESEARFHIDSVSEGNAGLYRCLYYKPPGWSEHSDFLELLVKESSGGPDSPDTEPGSSAGTVPGTEASGFDAPDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSGGGGSGGGGSGGGGSGIPPHVQKSVNNDMIVTDNNGAVKFPQLCKFCDVRFSTCDNQKSCMSNCSITSICEKPQEVCVAVWRKNDENITLETVCHDPKLPYHDFILEDAASPKCIMKEKKKPGETFFMCSCSSDECNDNIIFSEEYNTSNPD

SEQ ID NO: 32 - protein, 1038 aa, synthetic construct
  REGION 1..1038: note = IL12/LAIR2/IgG1Fc/TGF-Beta-RII
IWELKKDVYVVELDWYPDAPGEMVVLTCDTPEEDGITWTLDQSSEVLGSGKTLTIQVKEFGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLKYENYTSSFFIRDIIKPDPPKNLQLKPLKNSRQVEVSWEYPDTWSTPHSYFSLTFSVQVQGKSKREKKDRVFTDKTSATVICRKNASISVRAQDRYYSSSWSEWASVPCSGGGGSGGGGSGGGGSRNLPVATPDPGMFPCLHHSQNLLRAVSNMLQKARQTLEFYPCTSEEIDHEDITKDKTSTVEACLPLELTKNESCLNSRETSFITNGSCLASRKTSFMMALCLSSIYEDLKMYQVEFKTMNAKLLMDPKRQIFLDQNMLAVIDELMQALNFNSETVPQKSSLEEPDFYKTKIKLCILLHAFRIRAVTIDRVMSYLNASGGSGGQEGALPRPSISAEPGTVISPGSHVTFMCRGPVGVQTFRLEREDRAKYKDSYNVFRLGPSESEARFHIDSVSEGNAGLYRCLYYKPPGWSEHSDFLELLVKESSGGPDSPDTEPGSSAGTVPGTEASGFDAPDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSGGGGSGGGGSGGGGSGIPPHVQKSVNNDMIVTDNNGAVKFPQLCKFCDVRFSTCDNQKSCMSNCSITSICEKPQEVCVAVWRKNDENITLETVCHDPKLPYHDFILEDAASPKCIMKEKKKPGETFFMCSCSSDECNDNIIFSEEYNTSNPD

SEQ ID NO: 33 - protein, 893 aa, synthetic construct
  REGION 1..893: note = LAIR2/Fc(S254A)/IL-12PR
QEGALPRPSISAEPGTVISPGSHVTFMCRGPVGVQTFRLEREDRAKYKDSYNVFRLGPSESEARFHIDSVSEGNAGLYRCLYYKPPGWSEHSDFLELLVKESSGGPDSPDTEPGSSAGTVPGTEASGFDAPDKTHTCPPCPAPELLGGPSVFLFPPKPKDTLMIARTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSGGGGSGGGGSGGGGSIWELKKDVYVVELDWYPDAPGEMVVLTCDTPEEDGITWTLDQSSEVLGSGKTLTIQVKEFGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLKYENYTSSFFIRDIIKPDPPKNLQLKPLKNSRQVEVSWEYPDTWSTPHSYFSLTFSVQVQGKDNTEGRVFTDKTSATVICRKNASISVRAQDRYYSSSWSEWASVPCSGGGGSGGGGSGGGGSRNLPVATPDPGMFPCLHHSQNLLRAVSNMLQKARQTLEFYPCTSEEIDHEDITKDKTSTVEACLPLELTKNESCLNSRETSFITNGSCLASRKTSFMMALCLSSIYEDLKMYQVEFKTMNAKLLMDPKRQIFLDQNMLAVIDELMQALNFNSETVPQKSSLEEPDFYKTKIKLCILLHAFRIRAVTIDRMSYLNAS

SEQ ID NO: 34 - protein, 127 aa, synthetic construct [sequence omitted]

SEQ ID NO: 35 - protein, 157 aa, synthetic construct
  REGION 1..157: note = huFLT3L
TQDCSFQHSPISSDFAVKIRELSDYLLQDYPVTVASNLQDEELCGGLWRLVLAQRWMERLKTVAGSKMQGLLERVNTEIHFVTKCAFQPPPSCLRFVQTNISRLLQETSEQLVALKPWITRQNFSRCLELQCQPDSSTLPPPWSPRPLEATAPTAPQ

SEQ ID NO: 36 - protein, 227 aa, synthetic construct
  REGION 1..227: note = IgG1-Fc
KYGPPCPPCPAPEFEGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVTLWCLVKGFYPSDIAVEWESNGQPENNYKTTPPVLDSDGSFFLYSRLTVDKSRWQEGNVFSCSVMHEALHNHYTQKSLSLSLGK

SEQ ID NO: 37 - protein, 227 aa, synthetic construct
  REGION 1..227: note = IgG1-Fc-S254A/H435A (decrease FcRn binding) Could be single mutation
DKTHTCPPCPAPELLGGPSVFLFPPKPKDTLMIARTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNAYTQKSLSLSPGK

SEQ ID NO: 38 - protein, 226 aa, synthetic construct
  REGION 1..226: note = IgG1-Fc-YTE (improve FcRn binding)
KTHTCPPCPAPELLGGPSVFLFPPKPKDTLYITREPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 39 - protein, 247 aa, synthetic construct
  REGION 1..247: note = Alpha-PD-1 scFv (N-633-016-D23 scFv) VH/VL sequences never been disclosed previously
DIQMTQSPSSLSASVGDRVTITCQASQDISNYLNWYQQKPGKAPKLLIYDASNLETGVPSRFSGSGSGTDFTFTISSLQPEDIATYYCQQSTAFPFTFGQGTKVEIKGGGGSGGGGSGGGGSGGGGEVQLVESGGGLVQPGGSLRLSCAASGFTFSSYLMHWVRQAPGKGLEWVSVISLSGGSTYYADSVKGRFTISRDNSKNTLYLQMNSLRAEDTAVYYCARDRSSWALLGFDVWGQGTLVTVSS

SEQ ID NO: 40 - protein, 246 aa, synthetic construct
  REGION 1..246: note = Alpha-CTLA-4 scFv (aka-Alpha-CTLA-4-7 or N-644-007 scFv)
DIQMTQSPSSLSASVGDRVTITCQASQDISNYLNWYQQKPGKAPKLLIYDASNLETGVPSRFSGSGSGTDFTFTISSLQPEDIATYYCQQGDAWPWTFGQGTKVEIKGGGGSGGGGSGGGGSEVQLVESGGGLVQPGGSLRLSCAASGFTFTSYSMHWVRQAPGKGLEWVSGISGSGRSTSYADSVKGRFTISRDNSKNTLYLQMNSLRAEDTAVYYCARDLMAFNLVRAGGFDVWGQGTLVTVSS

SEQ ID NO: 41 - protein, 215 aa, synthetic construct
  REGION 1..215: note = N-601-Alpha-PD-L1 LC
NIQMTQSPSSVSASVGDRVTITCRASQDISRWLAWYQQKPGKAPKLLIYAASSLQSGVPSRFSGSGSGTDFALTISSLQPEDFATYYCQQADSRFSITFGQGTRLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 42 - protein, 448 aa, synthetic construct
  REGION 1..448: note = N-601-Alpha-PD-L1 HC
EVQLVQSGGGLVQPGGSLRLSCAASGFTFSSYSMNWVRQAPGKGLEWVSYISSSSSTIQYADSVKGRFTISRDNAKNSLYLQMNSLRDEDTAVYYCARGDYYYGMDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 43 - protein, 214 aa, synthetic construct
  REGION 1..214: note = TNT LC
EIVLTQSPATLSLSPGERATLSCRARQSISNYLHWYQQKPGQAPRLLIYYASQSISGIPDRFSGSGSGTDFTLTISRLEPEDFAVYYCQQSNSWPLTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 44 - protein, 451 aa, synthetic construct
  REGION 1..451: note = TNT HC
QVQLVQSGAEVKKPGASVKVSCKASGYTFTRYWMHWVRQAPGQGLEWIGAIYPGNSDTSYNQKFKGKATITADTSTNTAYMELSSLRSEDTAVYYCARGEEIGSRRWFAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 45 - protein, 99 aa, synthetic construct
  REGION 1..99: note = CCL2
MKVSAALLCLLLIAATFIPQGLAQPDAINAPVTCCYNFTNRKISVQRLASYRRITSSKCPKEAVIFKTIVAKEICADPKQKWVQDSMDHLDKQTQTPKT

SEQ ID NO: 46 - protein, 92 aa, synthetic construct
  REGION 1..92: note = CCL3
MQVSTAALAVLLCTMALCNQFSASLAADTPTACCFSYTSRQIPQNFIADYFETSSQCSKPGVIFLTKRSRQVCADPSEEWVQKYVSDLELSA

SEQ ID NO: 47 - protein, 92 aa, synthetic construct
  REGION 1..92: note = CCL4
MKLCVTVLSLLMLVAAFCSPALSAPMGSDPPTACCFSYTARKLPRNFVVDYYETSSLCSQPAVVFQTKRSKQVCADPSESWVQEYVYDLELN

SEQ ID NO: 48 - protein, 91 aa, synthetic construct
  REGION 1..91: note = CCL5
MKVSAAALAVILIATALCAPASASPYSSDTTPCCFAYIARPLPRAHIKEYFYTSGKCSNPAVVFVTRKNRQVCANPEKKWVREYINSLEMS

SEQ ID NO: 49 - protein, 98 aa, synthetic construct
  REGION 1..98: note = CCL19
MALLLALSLLVLWTSPAPTLSGTNDAEDCCLSVTQKPIPGYIVRNFHYLLIKDGCRVPAVVFTTLRGRQLCAPPDQPWVERIIQRLQRTSAKMKRRSS

SEQ ID NO: 50 - protein, 134 aa, synthetic construct
  REGION 1..134: note = CCL21
MAQSLALSLLILVLAFGIPRTQGSDGGAQDCCLKYSQRKIPAKVVRSYRKQEPSLGCSIPAILFLPRKRSQAELCADPKELWVQQLMQHLDKTPSPQKPAQGCRKDRGASKTGKKGKGSKGCKRTERSQTPKGP

(19) United States
(12) Patent Application Publication
(10) Pub. No.: US 2025/0042971 A1
(43) Pub. Date: Feb. 6, 2025

(54) LAIR-2 Compositions and Methods

(71) Applicant: ImmunityBio, Inc., Culver City, CA (US)

(72) Inventor: Patrick Soon-Shiong, Culver City, CA (US)

(73) Assignee: ImmunityBio, Inc., Culver City, CA (US)

(21) Appl. No.: 18/783,324

(22) Filed: Jul. 24, 2024

Related U.S. Application Data
(60) Provisional application No. 63/515,444, filed on Jul. 25, 2023.

(51) Int. Cl. C07K 14/705; A61K 39/00; A61P 35/00; C07K 14/71; C07K 16/24; C07K 16/28

(52) U.S. Cl. CPC C07K 14/70596; A61P 35/00; C07K 14/71; C07K 16/241; C07K 16/2827; A61K 2039/505; C07K 2319/30

(57) ABSTRACT

Provided herein are compositions and methods comprising Leukocyte-associated immunoglobulin-like receptor 2 (LAIR-2) and a Fc domain. Further provided herein are compositions and methods comprising TNT antibody fused to a TGFβ trap. The compositions and methods provided herein are useful for modulating a collagen matrix in a solid tumor.

This application claims the benefit of priority to U.S. Provisional Application No. 63/515,444 filed on Jul. 25, 2023, which is incorporated by reference in their entirety.

### SEQUENCE LISTING

The content of the XML file of the sequence listing named 1026900062PCT.xml, which is 81,460 bytes in size was created on Jul. 22, 2024 and electronically submitted via EFS-Web along with the present application, and is incorporated by reference in its entirety.

### FIELD OF THE INVENTION

The field of the invention is immunotherapy technologies.

### BACKGROUND

The background description includes information that may be useful in understanding the present disclosure. It is not an admission that any of the information provided herein is prior art or relevant to the presently claimed invention, or that any publication specifically or implicitly referenced is prior art.

The extracellular matrix (ECM) is a major structural component in all tissues. It comprises a non-cellular meshwork of proteins, glycoproteins, proteoglycans, and polysaccharides with collagens as the most abundant proteins. Ongoing ECM remodeling ensures tissue integrity and function, with specific collagens being synthesized and degraded in a highly regulated manner.

Several epithelial tumors including breast, pancreatic, colorectal, ovarian and lung cancer are characterized by a dense ECM where high collagen content correlates with poor prognosis. Cancer-associated fibroblasts (CAFs), macrophages and tumor cells themselves all contribute to increased collagen production and remodeling during cancer progression.

Most tumors overexpress a diverse set of collagens in an abnormal fashion. Overexpression of collagens has been associated with poor overall survival in several tumor types, such as lung, colorectal, and ovarian cancer. The capacity of tumors to induce remodeling of collagens in the tumor microenvironment (TME) was primarily thought to create a suitable microenvironment for tumor cell growth. However, recently there are reports that the abnormal collagen production, composition and organization in the TME may be a cause of immune dysfunction, conveying a chronic-active wound-healing response instead of anti-tumor immune responses necessary for immune surveillance and the eradication of the tumor. The abnormal ECM also builds physical fibrotic barriers to exclude immune cells and therapeutic agents from access to tumor cells. Ramos et al (2021) “Cancer immunotherapy by NC410, a LAIR-2 Fc protein blocking human LAIR-collagen interaction”, eLife 10:e62927.

TME-expressed collagen interacts with the inhibitory collagen receptor leukocyte-associated immunoglobulin-like receptor 1 (LAIR-1). LAIR-1 is an immune checkpoint broadly expressed on the cell surface of immune cells that binds to collagen and molecules with collagen-like domains. Upon triggering, LAIR-1 inhibits NK cell, T cell, B cell, monocyte, and DC function. Thus, besides formation of an ECM-rich and fibrotic tumor niche, TME-expressed collagens can function to promote immune evasion through their direct interaction with LAIR-1.

Thus, there remains a need in the art to block LAIR-1-mediated inhibition, such that immune cells can access the TME more efficiently.

### SUMMARY

The inventive subject matter provides compositions and methods for generating and using various fusion proteins in the treatment of a tumor. The fusion proteins, compositions and methods disclosed herein comprise leukocyte-associated immunoglobulin-like receptor 2 (LAIR-2), TNT-trap, and/or TGFβ. LAIR-2 is a natural soluble homolog of LAIR-1 that is present in humans and non-human primates. LAIR-2 shares 83% identity with the LAIR-1 extracellular region, binds to collagens with higher affinity than LAIR-1, and functions as a natural antagonist to block cell membrane LAIR-1 inhibitory signaling. The inventors have developed multiple fusion protein constructs comprising LAIR-2. Compositions containing the fusion protein constructs disclosed herein target tumor ECM and promote T cell function through blockade of LAIR-1-mediated inhibition. In terms of treating a tumor, the disclosed compositions were found to have an unexpected and synergistic effect in reducing the tumor growth in a patient.

In an embodiment, the inventive subject matter comprises a fusion protein comprising Leukocyte-associated immunoglobulin-like receptor 2 (LAIR-2) and an Fc domain. The Fc domain disclosed herein is selected from the group consisting of IgG1Fc (SEQ ID NO:16 or SEQ ID NO:36), IgG2Fc, and/or IgG4Fc. The fusion protein of the present disclosure may further comprise a TGFβ trap, optionally wherein the TGFβ trap is TGFβRII (SEQ ID NO: 18). The fusion protein may also comprise an immunostimulatory domain, optionally selected from the group consisting of IL-12 (SEQ ID NO: 19), IL-15, IL-15:IL-15RαSu, N-803, or an agonist derivative thereof. In another embodiment, disclosed herein is a fusion protein comprising a Tumor Necrosis Targeted (TNT) antibody fused to a TGFβ trap. Preferably, the TGFβ trap is TGFβRII. In one embodiment, the fusion protein has a polypeptide sequence with at least 80% sequence homogeneity to SEQ ID NO: 1 to SEQ ID NO:50.

In another aspect, the present disclosure provides a composition comprising a tumor-targeted antibody fused to a TGFβ trap, LAIR-2 or a fusion protein derivative thereof, and a checkpoint inhibitor antibody or binding domain thereof. The tumor targeted antibody is preferably a TNT antibody. The TGFβ trap is contemplated to be a TGFβ antibody, and/or soluble TGFβ receptor. Preferably, the tumor-targeted antibody fused to TGFβ trap has a polypeptide sequence with at least 80% sequence homogeneity to SEQ ID NO:3, or SEQ ID NO: 4. The LAIR-2 or fusion protein derivative thereof has a polypeptide sequence with at least 80% sequence homogeneity to a sequence selected from the list consisting of SEQ ID NO:1, SEQ ID NO:2, SEQ ID NO:5, SEQ ID NO:6, SEQ ID NO:7, SEQ ID NO:8, SEQ ID NO:9, SEQ ID NO: 10, SEQ ID NO:11, SEQ ID NO:15, SEQ ID NO:29, SEQ ID NO:30, SEQ ID NO:31, SEQ ID NO:32, and SEQ ID NO:33.

Preferably, LAIR-2 comprises a stabilized fusion protein derivative, such as wherein the LAIR-2 is fused to a tumor targeted antibody, binding domain thereof, or protein comprising a tumor-specific binding domain. Contemplated tumor targeted antibodies include a TNT antibody or binding domain thereof, and/or an anti-PD-L1 antibody or binding domain thereof. In further embodiments, the LAIR-2 is fused to an immunostimulatory domain, for stimulating T cells and/or Natural Killer (NK) cells, wherein the immunostimulatory domain comprises IL-12, IL-15, IL-15:IL-15RαSu, N803 or an agonist derivative thereof. The checkpoint inhibitor antibody comprises a PD-L1 antibody, a PD-1 antibody, or a CTLA-4 antibody. In preferred embodiments, the LAIR-2 is fused to the checkpoint inhibitor antibody or binding domain thereof, and/or the LAIR-2 is fused to the TGFβ trap fusion protein, and/or the checkpoint inhibitor antibody or binding domain thereof is fused to the TGFβ trap. In especially preferred embodiments, the checkpoint inhibitor antibody or binding domain thereof, LAIR-2, and the TGFβ trap are fused into a single soluble fusion protein complex. Furthermore, the checkpoint inhibitor binding domain comprises a CAR, and wherein the CAR is expressed by an NK cell. The composition may further comprise an anti-clusterin antibody. The above disclosed compositions modulate the collagen matrix in a solid tumor, and LAIR-2 releases LAIR-1-mediated immune suppression. This results in increased T cell and NK cell expansion and effector function, decreases tumor growth, and increasing the survival of patients having a tumor.

Further disclosed herein is a composition comprising a tumor-targeted antibody fused to a TGFβ trap, an HDAC inhibitor, and IL-15 or an agonist derivative thereof. In contemplated compositions, the tumor targeted antibody is a TNT antibody, and the TGFβ trap is a TGFβ antibody or a soluble TGFβ receptor. The HDAC inhibitor is contemplated to be a class I HDAC inhibitor, such as entinostat or zabadinostat. The IL-15 agonist derivative comprises IL15:IL 15RaSu or N-803. The above disclosed compositions modulate the collagen matrix in a solid tumor, and LAIR-2 releases LAIR-1-mediated immune suppression. This results in increased T cell and NK cell expansion and effector function, decreases tumor growth, and increasing the survival of patients having a tumor.

The present disclosure also contemplates a method of treating a patient having a solid tumor, by administering to the patient an effective amount of any one of the compositions disclosed herein. The treatment method may further comprise administering a T cell (such as CAR-T cell) and/or an NK cell, such as a N-92 cell or a genetically modified derivative thereof, or a primary NK cell (such as ceNK cell or m-ceNK cell). The treatment method may also include administering IL-15 or an agonist derivative thereof. The above disclosed compositions are administered to the patient intravenously, subcutaneously, intravesically, or intratumorally. The inventors have shown that administration of the above disclosed compositions results in collagen modification in the tumor microenvironment, and results in increased T and/or NK cells in the tumor.

In another aspect, the present disclosure provides a kit comprising a fusion protein or a composition disclosed above, and instructions for using the same.

Various objects, features, aspects and advantages of the inventive subject matter will become more apparent from the following detailed description of preferred embodiments, along with the accompanying drawing figures in which like numerals represent like components.

### BRIEF DESCRIPTION OF THE DRAWINGS

FIG. 1 illustrates the structure of N830 (TNT-TGFβRII) (also referred to herein as “TNT-Trap”) as well as its expression, purification, and ssDNA binding and TGFβ inhibition. The amino acid sequence of N830 is shown in SEQ ID NO:3 (light chain) and SEQ ID NO:4 (heavy chain respectively).

FIG. 2 illustrates the anti-tumor effect of TNT-trap in combination with a checkpoint inhibitor, anti-PD-L1 or anti-PD1.

FIG. 3 illustrates anti-tumor activity of TNT-Trap against EMT6.

FIG. 4 illustrates anti-tumor activity of TNT-Trap (N-830) in combination with N803 and Entinostat+/−aPDL1.

FIG. 5 illustrates anti-tumor activity of TNT-Trap+anti-PD-L1+NC410 (N410 is a fusion protein of LAIR-2 and Fc) on MC38 colon model. This combination was found to have a synergistic effect on tumor cell killing and tumor volume reduction.

FIG. 6 further illustrates anti-tumor activity of TNT-Trap+anti-PD-L1+NC410 on MC38 colon model. This combination was found to have a synergistic effect on tumor cell killing and tumor volume reduction.

FIG. 7 illustrates LAIR-2 constructs of the present disclosure.

FIG. 8 further illustrates LAIR-2 constructs of the present disclosure.

FIG. 9 illustrates LAIR-2 constructs, version 1, and shows the expression and HPLC elution profiles. The amino acid sequences corresponding to these constructions are SEQ ID NO: 5, SEQ ID NO:29, and SEQ ID NO:30 respectively.

FIG. 10 illustrates LAIR-2 constructs, version 1, and shows the expression and HPLC elution profiles. The amino acid sequences corresponding to these constructions are SEQ ID NO: 31 and SEQ ID NO:32 respectively.

FIG. 11 illustrates LAIR-2 constructs, version 2, and shows the expression and HPLC elution profiles. The amino acid sequences corresponding to these constructions are SEQ ID NO: 5, SEQ ID NO:6, and SEQ ID NO: 7 respectively.

FIG. 12 illustrates LAIR-2 constructs, version 3, and shows the expression and HPLC elution profiles. The amino acid sequences corresponding to these constructions are SEQ ID NO: 8, SEQ ID NO:33, SEQ ID NO: 10, and SEQ ID NO: 11 respectively.

FIG. 13 illustrates the FcRn binding sites on IgG1.

FIG. 14 illustrates the BLI binding experimental set up for huFcRn (human FcRn) and muFcRn (mouse FcRn) binding to LAIR-2/Fc*/IL-12 (PR).

FIG. 15 further illustrates the BLI binding experimental set up for huFcRn (human FcRn) and muFcRn (mouse FcRn) binding to LAIR-2/Fc*/IL-12 (PR).

FIG. 16 illustrates subcutaneous immunotherapy schedule with LAIR-2/Fc+TNT-TRAP+Anti-PD-L1 in MC38 colon tumor model.

### DETAILED DESCRIPTION

The inventive subject matter provides compositions and methods of using various fusion proteins featuring leukocyte-associated immunoglobulin-like receptor 2 (LAIR2), TNT-trap, and/or TGFβ. The compositions and methods disclosed herein re-model the Tertiary Lymphoid Structure (TLS) of a tumor, which in turn is useful for the treatment of the tumor. All tumors generally comprise a collagen matrix which is a barrier to immune cell invasion. LAIR-1 is expressed on immune cells and binds to collagen, thereby promoting immune cell invasion into a tumor while inhibiting the immune cell activity. LAIR-2 antagonistically binds to collagen, thereby leaving LAIR-1-expressing immune cells (T, NK, monocytes, B cells, DC) activated at the site of the tumor.

Collagens are a primary component of the extracellular matrix of tumors and are functional ligands for LAIR-1, which is expressed on immune cells. LAIR-2 is a secreted protein that can act as a decoy receptor by binding collagen with higher affinity than LAIR-1. The binding of LAIR2 to the collagen matrix releases LAIR1 expressing immune cells activated at the site of the tumor. In this disclosure, the inventors have shown that compositions comprising LAIR2 fusion proteins unexpectedly increase T cell and NK cell expansion and reduce tumor growth.

In one aspect, the present disclosure provides a fusion protein comprising LAIR-2 and a Fc domain. Fusion protein or fusion protein complex generally refers two or more proteins or protein domains that are covalently linked together, for example, by a peptide bond, disulfide bond, chemical cross-linking agent, etc. Often, but not necessarily, there is a linker between the two or more protein domains. The fusion protein complexes of the invention are multimerized, e.g., dimerized, trimerized, or otherwise multimerized (e.g., 4 complexes, 5 complexes, etc.). For example, the multimers are homomultimers or hetero-multimers. In some cases, one soluble fusion protein is covalently linked to another soluble fusion protein by a disulfide bond linking the Fc domain of the first soluble protein to the Fc domain of the second soluble protein.

The Fc domain or functional fragment thereof includes an Fc domain selected from the group consisting of IgG Fc domain, human IgG1 Fc domain, human IgG2 Fc domain, human IgG3 Fc domain, human IgG4 Fc domain, IgA Fc domain, IgD Fc domain, IgE Fc domain, and IgM Fc domain; mouse IgG2A domain, or any combination thereof. Optionally, the Fc domain includes an amino acid change that results in an Fc domain with altered complement or Fc receptor binding properties or altered dimerization or glycosylation profiles. Amino acid changes to produce an Fc domain with altered complement or Fc receptor binding properties or altered dimerization or glycosylation profiles are known in the art. For example, a substitution of leucine residues at positions 234 and 235 of the IgG1 CH2 (numbering based on antibody consensus sequence) (i.e., . . . . P E L L G G . . . ) with alanine residues (i.e., . . . . P E A A G G . . . ) results in a loss of Fc gamma receptor binding, whereas the substitution of the lysine residue at position 322 of the IgG1 CH2 (numbering based on antibody consensus sequence) (i.e., . . . . K C K S L . . . ) with an alanine residue (i.e., . . . . K C A S L . . . ) results in a loss of complement activation. In some examples, such mutations are combined.

The fusion protein comprising LAIR-2 and Fc domain as disclosed herein may comprise an IgG1 Fc domain, an IgG2 Fc domain, and/or an IgG4 Fc domain. The fusion protein may further comprise a TGFβ trap. In one embodiment, the TGFβ trap is TGFβRII.

The fusion protein comprising LAIR-2 and Fc domain as disclosed herein may further comprise an immunostimulatory domain. The immunostimulatory domain may be IL-12, IL-15, IL-15:IL-15RαSu, or an agonist derivative thereof. The IL-15 agonist derivative is preferably N-803. U.S. Pat. Nos. 11,129,883B2 and US20220033455A1, the contents of which are incorporated by reference in its entirety, discusses the specific structures of the various immunostimulatory domains.

The present disclosure further provides a fusion protein comprising a Tumor Necrosis Targeted (TNT) antibody fused to a TGFβ trap. In this regard, humanized TNT antibodies for targeting necrosis in cancer therapy is disclosed in the U.S. Pat. No. 11,384,157B2, the contents of which is incorporated by reference in its entirety. In preferred embodiments, the TGFβ trap is TGFβRII. This fusion protein is useful for treating tumor because the tumor targeted antibody (TNT) fused to a TGFβ trap will clear TGFβ from the tumor, thereby activating cells of the TLS (Tertiary Lymphoid Structure). An antibody derived from the fusion protein comprising a TNT antibody fused to a TGFβ trap is also referred to herein as N830 or TNT-Trap. The SEQ ID NO: 3 and SEQ ID NO:4 illustrates the light chain and heavy chain of the N830 (TNT-trap) antibody.

SEQ ID NO: 1-50 provides the sequences of the above referenced fusion proteins, or protein domains thereof. The fusion proteins of the present disclosure may comprise polypeptide sequences having at least 60%, or at least 70%, or at least 80%, or at least 85%, or at least 90%, or at least 95%, or up to 100%, sequence homogeneity to SEQ ID NO: 1 to SEQ ID NO:50.

In another aspect, the present disclosure provides a composition comprising a tumor-targeted antibody fused to a TGFβ trap, a LAIR-2 or a fusion protein derivative thereof, an immunostimulatory protein (i.e. IL-12), and a checkpoint inhibitor antibody or binding domain thereof. In some embodiments, the composition disclosed herein modulates a collagen matrix in a solid tumor.

In yet another aspect, the present disclosure provides a composition comprising a tumor-targeted antibody fused to a TGFβ trap, a histone deacetylase (HDAC) inhibitor, and an IL-15 or an agonist derivative thereof. In some embodiments, the composition disclosed herein modulates a collagen matrix in a solid tumor. While all known HDAC inhibitors are contemplated, in preferred embodiments, the HDAC inhibitor is a class I HDC inhibitor such as entinostat.

The tumor targeted antibody may comprise any known tumor targeting or tumor specific antibody. In a preferred embodiment, the tumor targeting antibody is a TNT antibody. The TGFβ trap is preferably a TGFβ antibody and/or a soluble TGFβ receptor. The tumor-targeted antibody fused to TGFβ trap may comprise polypeptide sequences having at least 60%, or at least 70%, or at least 80%, or at least 85%, or at least 90%, or at least 95%, or up to 100%, sequence homogeneity to SEQ ID NO:3 and SEQ ID NO:4.

Additionally, the tumor targeted antibody may include various tumor-specific antibody domains known in the art. The antibodies and their respective targets for treatment of cancer include but are not limited to nivolumab (anti-PD-1 Ab), TA99 (anti-gp75), 3F8 (anti-GD2), 8H9 (anti-B7-H3), abagovomab (anti-CA-125 (imitation)), adecatumumab (anti-EpCAM), afutuzumab (anti-CD20), alacizumab pegol (anti-VEGFR2), altumomab pentetate (anti-CEA), amatuximab (anti-mesothelin), AME-133 (anti-CD20), anatumomab mafenatox (anti-TAG-72), apolizumab (anti-HLA-DR), arcitumomab (anti-CEA), bavituximab (anti-phosphatidylserine), bectumomab (anti-CD22), belimumab (anti-BAFF), besilesomab (anti-CEA-related antigen), bevacizumab (anti-VEGF-A), bivatuzumab mertansine (anti-CD44 v6), blinatumomab (anti-CD19), BMS-663513 (anti-CD137), brentuximab vedotin (anti-CD30 (TNFRSF8)), cantuzumab mertansine (anti-mucin CanAg), cantuzumab ravtansine (anti-MUC1), capromab pendetide (anti-prostatic carcinoma cells), carlumab (anti-MCP-1), catumaxomab (anti-EpCAM, CD3), cBR96-doxorubicin immunoconjugate (anti-Lewis-Y antigen), CC49 (anti-TAG-72), cedelizumab (anti-CD4), Ch. 14.18 (anti-GD2), ch-TNT (anti-DNA associated antigens), citatuzumab bogatox (anti-EpCAM), cixutumumab (anti-IGF-1 receptor), ivatuzumab tetraxetan (anti-MUC1), conatumumab (anti-TRAIL-R2), CP-870893 (anti-CD40), dacetuzumab (anti-CD40), daclizumab (anti-CD25), dalotuzumab (anti-insulin-like growth factor I receptor), daratumumab (anti-CD38 (cyclic ADP ribose hydrolase)), demcizumab (anti-DLL4), detumomab (anti-B-lymphoma cell), drozitumab (anti-DR5), duligotumab (antiHER3), dusigitumab (anti-ILGF2), ecromeximab (anti-GD3 ganglioside), edrecolomab (anti-EpCAM), elotuzumab (anti-SLAMF7), elsilimomab (anti-IL-6), enavatuzumab (anti-TWEAK receptor), enoticumab (anti-DLL4), ensituximab (anti-5AC), epitumomab cituxetan (anti-episialin), epratuzumab (anti-CD22), ertumaxomab (anti-HER2/neu, CD3), etaracizumab (anti-integrin av 3), faralimomab (anti-Interferon receptor), farletuzumab (anti-folate receptor 1), FBTA05 (anti-CD20), ficlatuzumab (anti-HGF), figitumumab (anti-IGF-1 receptor), flanvotumab (anti-TYRP1 (glycoprotein 75)), fresolimumab (anti-TGF™), futuximab (anti-EGFR), galiximab (anti-CD80), ganitumab (anti-IGF-1), gemtuzumab ozogamicin (anti-CD33), girentuximab (anti-carbonic anhydrase 9 (CA-IX)), glembatumumab vedotin (anti-GPNMB), guselkumab (anti-IL13), ibalizumab (anti-CD4), ibritumomab tiuxetan (anti-CD20), icrucumab (anti-VEGFR-1), igovomab (anti-CA-125), IMAB362 (anti-CLDN18.2), IMC-CS4 (anti-CSFIR), IMC-TR1 (TGF^{˜}RII), imgatuzumab (anti-EGFR), inclacumab (anti-selectin P), indatuximab ravtansine (anti-SDC1), inotuzumab ozogamicin (anti-CD22), intetumumab antiCD51), ipilimumab (anti-CD152), iratumumab (anti-CD30 (TNFRSF8)), KM3065 (anti-CD20), KW-0761 (anti-CD194), LY2875358 (anti-MET) labetuzumab (anti-CEA), lambrolizumab (antiPDCD1), lexatumumab (anti-TRAIL-R2), lintuzumab (anti-CD33), lirilumab (anti-KIR2D), lorvotuzumab mertansine (anti-CD56), lucatumumab (anti-CD40), lumiliximab (anti-CD23 (IgE receptor)), mapatumumab (anti-TRAIL-R1), margetuximab (anti-ch4D5), matuzumab (anti-EGFR), mavrilimumab (anti-GMCSF receptor α-chain), milatuzumab (anti-CD74), minretumomab (anti-TAG-72), mitumomab (anti-GD3 ganglioside), mogamulizumab (antiCCR4), moxetumomab pasudotox (anti-CD22), nacolomab tafenatox (anti-C242 antigen), naptumomab estafenatox (anti-5T4), narnatumab (anti-RON), necitumumab (anti-EGFR), nesvacumab (anti-angiopoietin 2), nimotuzumab (anti-EGFR), nivolumab (anti-IgG4), nofetumomab merpentan, ocrelizumab (anti-CD20), ocaratuzumab (anti-CD20), olaratumab (anti-PDGF-R a), onartuzumab (anti-c-MET), ontuxizumab (anti-TEM1), oportuzumab monatox (anti-EpCAM), oregovomab (anti-CA-125), otlertuzumab (anti-CD37), pankomab (anti-tumor specific glycosylation of MU CI), parsatuzumab (anti-EGFL7), pascolizumab (anti-IL-4), patritumab (anti-HER3), pemtumomab (anti-MUC1), pertuzumab (anti-HER2/neu), pidilizumab (anti-PD-1), pinatuzumab vedotin (anti-CD22), pintumomab (anti-adenocarcinoma antigen), polatuzumab vedotin (anti-CD79B), pritumumab (anti-vimentin), PRO131921 (anti-CD20), quilizumab (anti-IGHE), racotumomab (anti-N-glycolylneuraminic acid), radretumab (anti-fibronectin extra domain-B), ramucirumab (anti-VEGFR2), rilotumumab (anti-HGF), robatumumab (anti-IGF-1 receptor), roledumab (anti-RFID), rovelizumab (anti-CD11 & CD18), samalizumab (anti-CD200), satumomab pendetide (anti-TAG-72), seribantumab (anti-ERBB3), SGN-CD19A (anti-CD19), SGN-CD33A (anti-CD33), sibrotuzumab (anti-PAP), siltuximab (anti-IL-6), solitomab (anti-EpCAM), sontuzumab (anti-episialin), tabalumab (anti-BAFF), tacatuzumab tetraxetan (anti-alpha-fetoprotein), taplitumomab paptox (anti-CD19), telimomab aritox, tenatumomab (anti-tenascin C), teneliximab (anti-CD40), teprotumumab (anti-CD221), TGN1412 (anti-CD28), ticilimumab (anti-CTLA-4), tigatuzumab (anti-TRAIL-R2), TNX-650 (anti-IL-13), tositumomab (anti-CS20), tovetumab (anti-CD140a), TRBS07 (anti-GD2), tregalizumab (anti-CD4), tremelimumab (anti-CTLA-4), TRU-016 (anti-CD37), tucotuzumab celmoleukin (anti-EpCAM), ublituximab (anti-CD20), urelumab (anti-4-1BB), vantictumab (anti-Frizzled receptor), vapaliximab (anti-AOC3 (VAP-1)), vatelizumab (anti-ITGA2), veltuzumab (anti-CD20), vesencumab (anti-NRP1), visilizumab (anti-CD3), volociximab (antiintegrin a5B1), vorsetuzumab mafodotin (anti-CD70), votumumab (anti-tumor antigen CTAA16.88), zalutumumab (anti-EGFR), zanolimumab (anti-CD4), zatuximab (anti-HER1), ziralimumab (anti-CD147 (basigin)), RG7636 (anti-ETBR), RG7458 (anti-MUC16), RG7599 (anti-NaPi2b), MPDL3280A (anti-PD-L1), RG7450 (anti-STEAP1), and GDC-0199 (anti-Bcl-2). The tumor targeted antibody may be a bi-specific antibody, wherein at least one binding domain is specific for a tumor.

In one embodiment, the LAIR-2 or a fusion protein derivative thereof has a polypeptide sequence with at least 80% sequence homogeneity to a sequence selected from the list consisting of SEQ ID NO: 1, SEQ ID NO:2, SEQ ID NO:5, SEQ ID NO:6, SEQ ID NO: 7, SEQ ID NO:8, SEQ ID NO:9, SEQ ID NO: 10, SEQ ID NO:11, SEQ ID NO: 15, SEQ ID NO:29, SEQ ID NO: 30, SEQ ID NO:31, SEQ ID NO:32, and SEQ ID NO:33.

In certain embodiments, the LAIR-2 protein has a polypeptide sequence having at least 60%, or at least 70%, or at least 80%, or at least 85%, or at least 90%, or at least 95%, or up to 100%, sequence homogeneity to SEQ ID NO: 15. The LAIR-2 protein may further be stabilized as a fusion protein derivative. For example, the fusion protein derivative may comprise LAIR-2 fused to a tumor targeted antibody, binding domain thereof, or protein comprising a tumor-specific binding domain. In these cases, the any known tumor targeting antibody is contemplated, and preferred embodiments include a TNT antibody or binding domain thereof, an anti-PD-L1 antibody or binding domain thereof. In further embodiments, the LAIR-2 is fused to an immunostimulatory domain, for stimulating T cells and/or Natural Killer (NK) cells. The immunostimulatory domain may comprise IL-12, IL-15, IL-15:IL-15RαSu, or an agonist derivative thereof. In preferred embodiments, the IL-15 agonist derivative is IL-15:IL-15RαSu or N-803.

The checkpoint inhibitor, as disclosed in the present compositions and methods, inhibits the immune checkpoint suppressive activity or acts as an agonist of immune stimulatory activity. Such immune checkpoint and signaling molecules and ligands include PD-1, PD-L1, PD-L2, CTLA-4, CD28, CD80, CD86, B7-H3, B7-H4, B7-H5, ICOS-L, ICOS, BTLA, CD137L, CD137, HVEM, KM, 4-1BB, OX40L, CD70, CD27, CD47, CIS, OX40, GITR, IDO, TIM3, GAL9, VISTA, CD155, TIGIT, LIGHT, LAIR-1, SLAMF7, Siglecs and A2aR (Pardoll D M. 2012. Nature Rev Cancer 12:252-264, Thaventhiran T, et al. 2012. J Clin Cell Immunol S12:004). Additionally, preferred antibody domains of the invention may include ipilimumab and/or tremelimumab (anti-CTLA4), nivolumab, pembrolizumab, pidilizumab, TSR-042, ANB011, AMP-514 and AMP-224 (a ligand-Fc fusion) (anti-PD1), atezolizumab (MPDL3280A), avelumab (MSB0010718C), durvalumab (MEDI4736), MEDI0680, and BMS-9365569 (anti-PDL1), MEDI6469 (anti-OX40 agonist), BMS-986016, IMP701, IMP731, IMP321 (anti-LAG3) and GITR ligand. In especially preferred embodiments, the checkpoint inhibitor include a PD-L1 antibody, a PD-1 antibody, or a CTLA-4 antibody. In further embodiments, the checkpoint inhibitor binding domain comprises a CAR, and wherein the CAR is expressed by an NK cell.

In some embodiments, the presently disclosed compositions further comprise an anti-clusterin antibody.

In the compositions and methods of this disclosure, the LAIR-2 may be fused to the checkpoint inhibitor antibody or binding domain thereof. The LAIR-2 may further be fused to the TGFβ trap fusion protein. Alternatively or additionally, the checkpoint inhibitor antibody or binding domain thereof is fused to the TGFβ trap. In further embodiments, the checkpoint inhibitor antibody or binding domain thereof, the LAIR-2, and the TGFβ trap are fused into a single soluble fusion protein complex.

The inventors have also disclosed a method of treating a patient having a solid tumor, comprising administering to the patient an effective amount of a composition comprising one of more of the fusion proteins of this disclosure. Administration of the fusion protein complexes of the invention induces an immune response in a patient having a solid tumor. For example, administration of the fusion protein complexes induces an immune response against tumor cells. The fusion protein complex of the invention results in collagen modification in the tumor microenvironment, and/or results in increased T and or NK cells in the tumor.

The inventors have further disclosed a method of treating a patient having a solid tumor, comprising administering to the patient an effective amount of one or more of the compositions disclosed herein. The therapeutic compositions disclosed herein may further comprise a T cell or an NK cell, and more preferably a CAR-T cell or CAR-NK cell. The NK cell is preferably an N-92 cell, a genetically modified derivative thereof, or a primary NK cell. The primary NK cell may be a cytokine enhanced NK (ceNK) cell or a memory-like ceNK (m-ceNK) cell. The therapeutic methods disclosed herein may further comprise administering IL-15 or an agonist derivative thereof. The compositions of the invention disclosed herein results in collagen modification in the tumor microenvironment, and/or results in increased T and or NK cells in the tumor.

Solid tumors that are suitable for treatment with the methods and compositions described herein comprise sarcomas and carcinomas such as, but not limited to: fibrosarcoma, myxosarcoma, liposarcoma, chondrosarcoma, osteogenic sarcoma, chordoma, angiosarcoma, endotheliosarcoma, lymphangiosarcoma, lymphangioendotheliosarcoma, synovioma, mesothelioma, Ewing's tumor, leiomyosarcoma, rhabdomyosarcoma, colon carcinoma, pancreatic cancer, breast cancer, ovarian cancer, prostate cancer, squamous cell carcinoma, basal cell carcinoma, adenocarcinoma, sweat gland carcinoma, sebaceous gland carcinoma, papillary carcinoma, papillary adenocarcinomas, cystadenocarcinoma, medullary carcinoma, bronchogenic carcinoma, renal cell carcinoma, hepatoma, bile duct carcinoma, choriocarcinoma, seminoma, embryonal carcinoma, Wilms' tumor, cervical cancer, testicular tumor, lung carcinoma, small cell lung carcinoma, bladder carcinoma, epithelial carcinoma, glioma, astrocytoma, medulloblastoma, craniopharyngioma, ependymoma, pinealoma, hemangioblastoma, acoustic neuroma, oligodendroglioma, meningioma, melanoma, neuroblastoma, and retinoblastoma.

The pharmaceutical compositions comprising one or more fusion proteins as disclosed herein is administered in an effective amount to treat the tumor. For example, an effective amount of the pharmaceutical composition is between about 1 μg/kg and 100 μg/kg, e.g., 1, 5, 10, 15, 20, 25, 30, 35, 40, 45, 50, 55, 60, 65, 70, 75, 80, 85, 90, 95, or 100 μg/kg. Alternatively, the fusion protein complex is administered as a fixed dose or based on body surface area (i.e., per m²).

The pharmaceutical composition comprising the one or more fusion protein complexes is administered at least one time per month, e.g., twice per month, once per week, twice per week, once per day, twice per day, every 8 hours, every 4 hours, every 2 hours, or every hour. Suitable modes of administration for the adoptively transferred immune cells include systemic administration, intravenous administration, intratumoral administration, or local administration. Suitable modes of administration for the pharmaceutical composition include systemic administration, intravenous administration, local administration, subcutaneous administration, intramuscular administration, intratumoral administration, intraperitoneal administration, and intravesical administration.

The present disclosure is also directed towards kits or pharmaceutical systems comprising the compositions disclosed herein and instructions for using the same. Pharmaceutical compositions comprising the fusion protein complex and compositions of the invention are assembled into kits or pharmaceutical systems for use in ameliorating a tumor. Kits or pharmaceutical systems according to this aspect of the invention comprise a carrier means, such as a box, carton, tube, having in close confinement therein one or more container means, such as vials, tubes, ampoules, bottles and the like. The kits or pharmaceutical systems of the invention may also comprise associated instructions for using the fusion protein complexes and compositions of the invention. In one embodiment, the kit includes appropriate containers such as bags, bottles, tubes, to allow administration of such cells to a patient. Kits may also include medical devices comprising the fusion protein complex of the invention.

It is also contemplated that the pharmaceutical composition may comprise T-cells and/or NK cells. The T cells and/or NK cells contemplated herein may be from a tumor or from a blood sample of the patient, preferably modified, and expanded ex-vivo. The thusly expanded T cells and/or NK cells are reintroduced to the patient as autologous cells or administered to a different subject as donor cells or allogenic cells.

The T cells and/or NK cells are expanded by exposure to a cytokine cocktail comprising one or more of IL-2, IL-15, IL-12, IL-18, IL-21, and IL-7, or an agonist derivative thereof. Alternatively, the NK cells are genetically modified to express endoplasmic reticulum localized IL-15 (erIL-15). In this case, the genetic modification of the NK cells comprises introducing into the NK cell a nucleic acid that encodes a cytokine such as IL-2 or IL-15. The IL-2 may be expressed with a signal sequence that directs the IL-2 to the endoplasmic reticulum IL-2 (“erIL-2”). Similarly, the IL-15 may be expressed with a signal sequence that directs the IL-15 to the endoplasmic reticulum IL-15 (“erIL-15”). This permits expression of IL-2 and/or IL-15 at levels sufficient for autocrine activation, but without releasing IL-2 extracellularly. See Konstantinidis et al “Targeting IL-2 to the endoplasmic reticulum confines autocrine growth stimulation to NK-92 cells “Exp Hematol. 2005 February; 33 (2): 159-64

Furthermore, in some embodiments, T cells and/or NK cells are genetically modified to express a chimeric antigen receptor (CAR), and wherein the CAR targets a tumor antigen or a checkpoint inhibitor. The intracellular signaling domain of the CAR may also comprise an FcεRIγ portion. U.S. patent application Ser. No. 17/341,098, which discloses such methods, is herein incorporated by reference.

With respect to NK cells, it should be noted that all NK cells are deemed suitable for use herein and therefore include primary NK cells (preserved, expanded, and/or fresh cells), secondary NK cells that have been immortalized, autologous or heterologous NK cells (banked, preserved, fresh, etc.), and modified NK cells as described in more detail below. In some embodiments, it is preferred that the NK cells are NK-92 cells. The NK-92 cell line is a unique cell line that was discovered to proliferate in the presence of interleukin 2 (IL-2) (see e.g., Gong et al., Leukemia 8:652-658 (1994)). NK-92 cells are cancerous NK cells with broad anti-tumor cytotoxicity and predictable yield after expansion in suitable culture media. Advantageously, NK-92 cells have high cytolytic activity against a variety of cancers. Representative NK-92 cells are deposited with the American Type Culture Collection (ATCC), designation CRL-2407.

The NK-92 may be modified to express the high-affinity Fcγ receptor (CD16). Sequences for high-affinity variants of the Fcγ receptor are well known in the art (see e.g., Blood 2009 113:3716-3725), and all manners of generating and expression are deemed suitable for use herein. Expression of such receptor is believed to allow specific targeting of tumor cells using antibodies that are specific to a patient's tumor cells (e.g., neoepitopes), a particular tumor type (e.g., her2neu, PSA, PSMA, etc.), or that are associated with cancer (e.g., CEA-CAM). Advantageously, such antibodies are commercially available and can be used in conjunction with the cells (e.g., bound to the Fcγ receptor). Alternatively, such cells may also be commercially obtained from NantKwest as haNK cells. Such cells may then be additionally genetically modified to a CAR as further described in more detail below. U.S. Pat. Nos. 10,738,279, 10,456,420, 10,736,921, 11,000,550, 10,801,013, and 10,774,310

Where the NK cell is an autologous NK cell or an NK-92 cell it is contemplated that the recombinant nucleic acid will include a segment that encodes a CAR that includes FcεRIγ signaling domain, and preferably also a segment that encodes a cytokine to provide autocrine growth stimulation (e.g., IL-2, IL-2 that is modified with an ER retention sequence, IL-15, or IL-15 that is modified with an ER retention sequence) and/or a segment that encodes a CD16 or high affinity CD16-158V. As will be readily appreciated, inclusion of a cytokine that provides autocrine growth stimulation will render the modified recombinant independent of exogenous cytokine addition, which will render large scale production of such cells economically feasible. Likewise, where the modified recombinant also expresses CD16 or a high affinity CD16-158V, such cells will have further enhanced ADCC characteristics and with that further improved targeted cytotoxicity.

Contemplated CARs include a general structure of a desired antigen binding domain that is coupled to a hinge domain, which is coupled to a transmembrane domain, which is coupled to a signaling domain. The extracellular binding domain of the CAR is a scFv or other natural or synthetic binding portion that specifically binds an antigen of interest. Especially suitable binding portions include those that will specifically bind to a tumor-specific antigen, a tumor associated antigen, or a patient- and tumor-specific antigen. Tumor-specific antigens include, without limitation, NKG2D ligands, CS1, GD2, CD138, EpCAM, EBNA3C, GPA7, CD244, CA-125, ETA, MAGE, CAGE, BAGE, HAGE, LAGE, PAGE, NY-SEO-1, GAGE, CEA, CD52, CD30, MUC5AC, c-Met, EGFR, FAP, WT-1, PSMA, NY-ESO1, AFP, CEA, CTAGIB, and CD33. Still further tumor-specific antigens are described, by way of non-limiting example, in US2013/0189268; WO 1999024566 A1; U.S. Pat. No. 7,098,008; and WO 2000020460, each of which is incorporated herein by reference in its entirety.

The cytokine enhanced NK (ceNK) cells disclosed herein refers to NK cells in which the cytotoxic activity is enhanced by cytokine stimulation. ceNK cells are prepared by inducing NK cells with a corticosteroid and optionally a cytokine composition comprising IL-15, IL-15:IL-15Rα, or agonist derivatives thereof, such as N-803. The cytokine composition may comprise a fusion protein, wherein the fusion protein comprises IL-15 or an agonist derivative thereof. Fusion proteins comprising IL-15, wherein the fusion protein has increased stability over IL-15 are preferred. It is generally preferred that the corticosteroid is hydroxycortisone and the optional cytokine is N-803.

The Memory-like Cytokine-Enhanced NK Cells (m-ceNK) disclosed herein comprises enriched and expanded NK cells obtained from peripheral blood of donors using the apheresis technique to generate NK cells with a memory-like phenotype. The m-ceNK cells exhibit both high cytotoxicity and increased interferon-gamma (IFNγ) production. These m-ceNK cells can be generated from an individual donor for autologous cell therapy or may be generated as an allogeneic product from cord blood. In addition to the enhanced efficacy, m-ceNK cells can be infused easily in an outpatient setting.

Further description of making the m-ceNK cells and its advantageous properties is described in PCT/US2022/018290, which is incorporated by reference in its entirety. U.S. patent application Ser. No. 17/375,985 and U.S. Pat. No. 11,453,862 provide additional alternative methods of inducing enrichment and expansion of NK cells. Each of the above references are incorporated by reference in its entirety.

### EXAMPLES

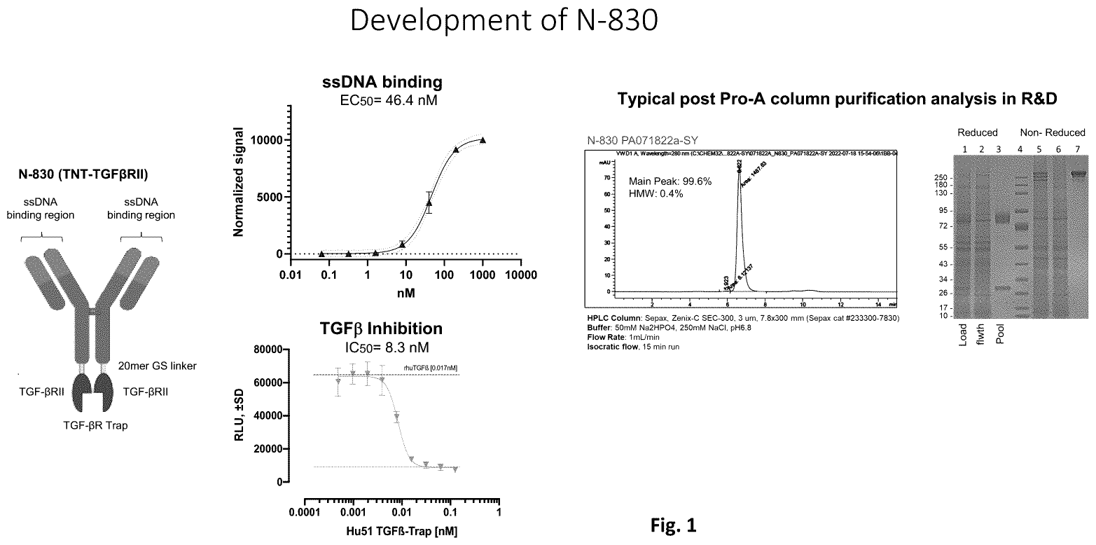

Example 1: Development of N-830 (TNT-Trap). The term N-830 as used herein refers to a protein complex comprising TNT antibody and TGFβRII. The polypeptide sequences of the light and heavy chain of N830 are disclosed in SEQ ID NO:3 and SEQ ID NO:4 respectively. FIG. 1 illustrates the development of N830. A Pro-A column purification shows that the antibody elutes as one peak, illustrating that there is no decomposition or breakdown of the antibody complex. The ssDNA binding data and the TGFβ inhibition data shows that the N830 is active.

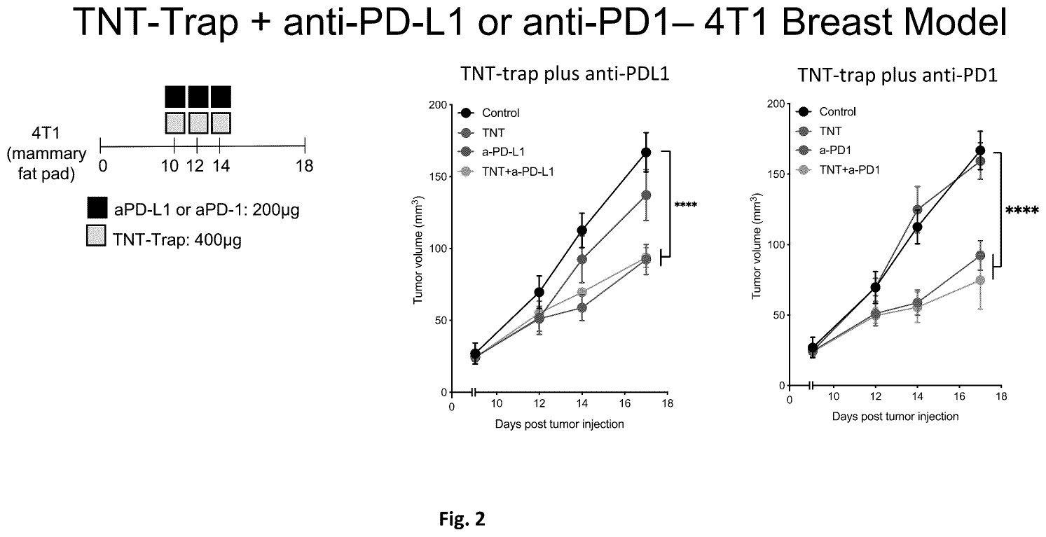

Example 2: FIG. 2 shows selected in-vivo data on N-830 (TNT-Trap). TNT-Trap+anti-PD-L1 or anti-PD1 were used on 4T1 Breast Model. The 4T1 model is rich in TGFβ. This experiment was aimed at investigating a possible advantage of the combination of TNT-Trap with checkpoint inhibition. 4T1 (5×10⁴) cells were orthotopically injected into the mammary fat pad of female adult BALB/C mice. Intraperitoneal injections of anti PD-L1 (200 ug) and N-830 (400 ug) alone or in combination were given on days 10, 12, and 14. Tumor volumes were measured every 2 to 3 days. TNT-trap monotherapy was found to have significant anti-tumor effect in the 4T1 Model, as shown in FIG. 2.

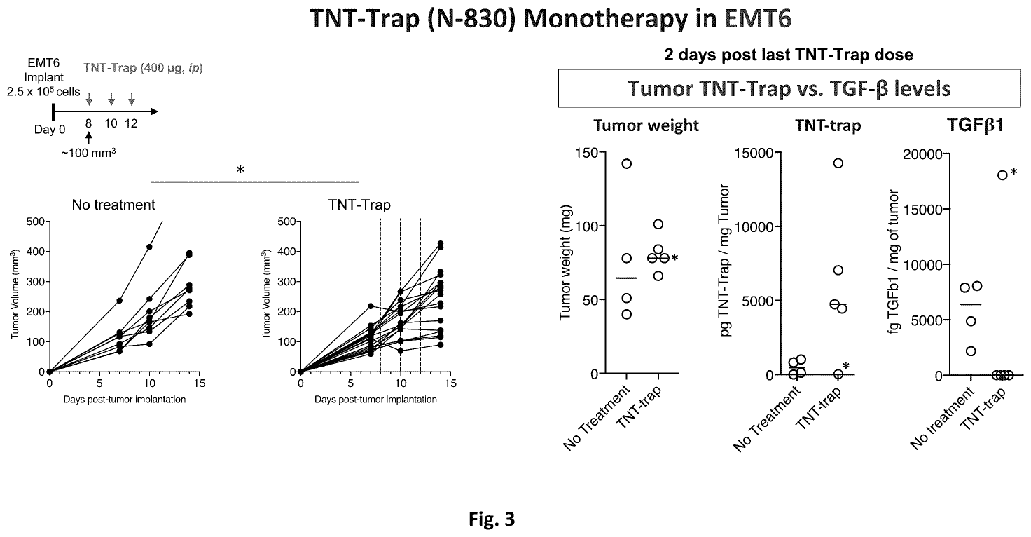

Example 3: TNT-Trap (N-830) Monotherapy was used on in EMT6 cells. BALB/c mice were inoculated s.c. in the flank with 2.5×10⁵ EMT6 cells. Intraperitoneal of TNT-trap (400 μg) were given on days 8, 10, and 12. Tumor volumes were measured every 3 to 4 days. The results, shown on FIG. 3, illustrate that TNT-Trap shows mild signs of antitumor activity against EMT6. Moreover, TNT-trap was present in significant quantities in the TME two days after the last dose, which correlates with TGFβ1 levels below detection limit.

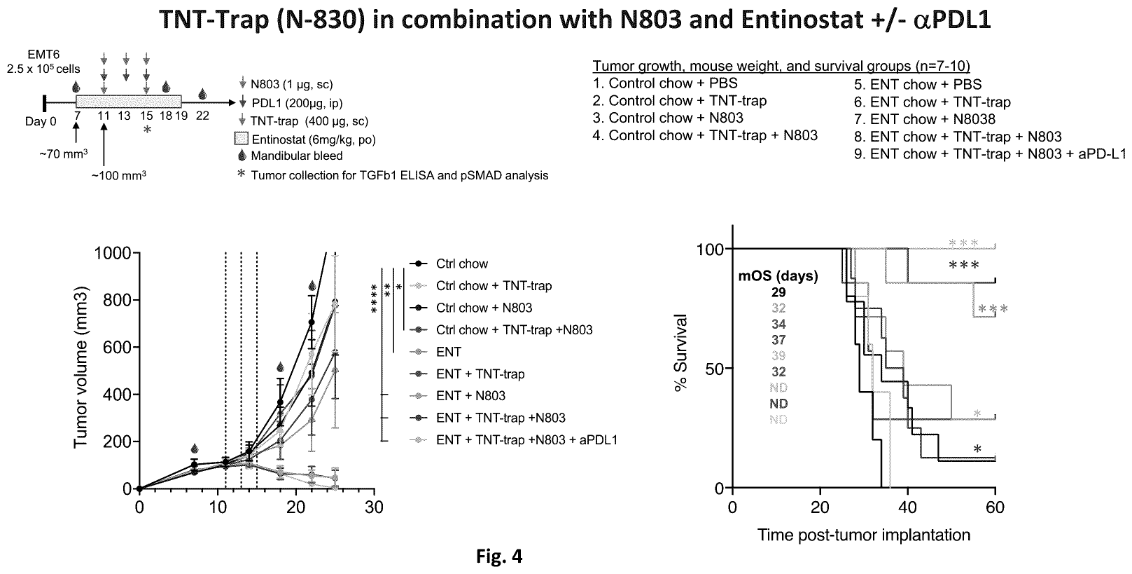

Example 4: TNT-Trap (N-830) in combination with N803 and Entinostat+/−aPDL1 were used on EMT6 cells. BALB/c mice were inoculated s.c. in the flank with 2.5×10⁵ EMT6 cells. Mice received control chow or Entinostat diet from day 7 and day 19. Intraperitoneal injections of anti-PD-L1 (200 μg) and subcutaneous injections of TNT-trap (400 μg) were given on days 11, 13, and 15. Subcutaneous injection of N803 (1 μg) were given on days 11 and 15. Tumor volumes were measured every 1 to 2 days. The results, shown on FIG. 4, illustrate that two combinations (i) TNT-Trap (N-830), N803, and Entinostat, and (ii) TNT-Trap (N-830), N803, Entinostat, and aPDL1, showed unexpectedly good results in reducing tumor volume. Thus the combination of the above four components leads synergistic results

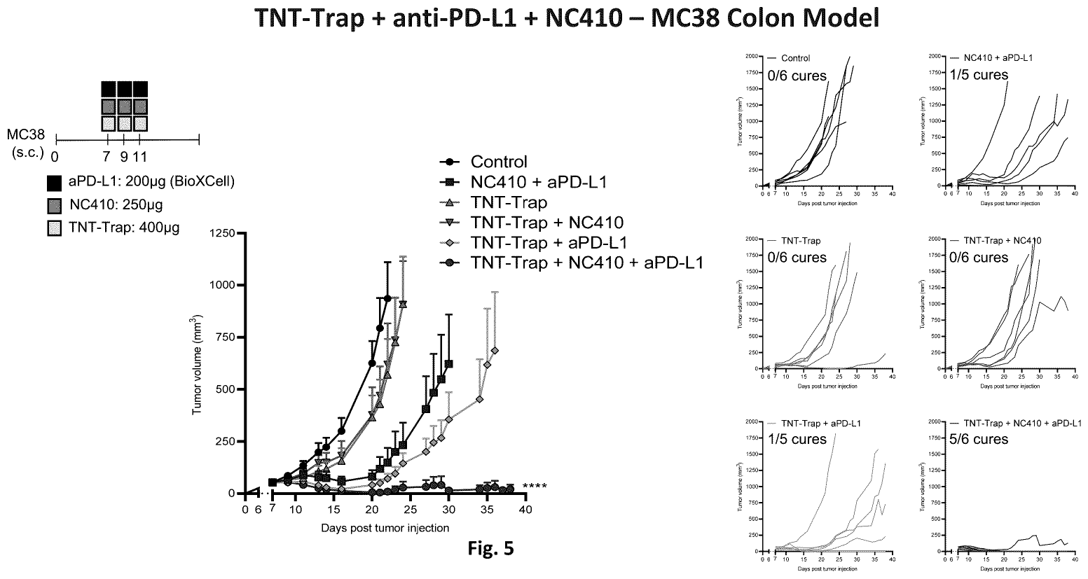

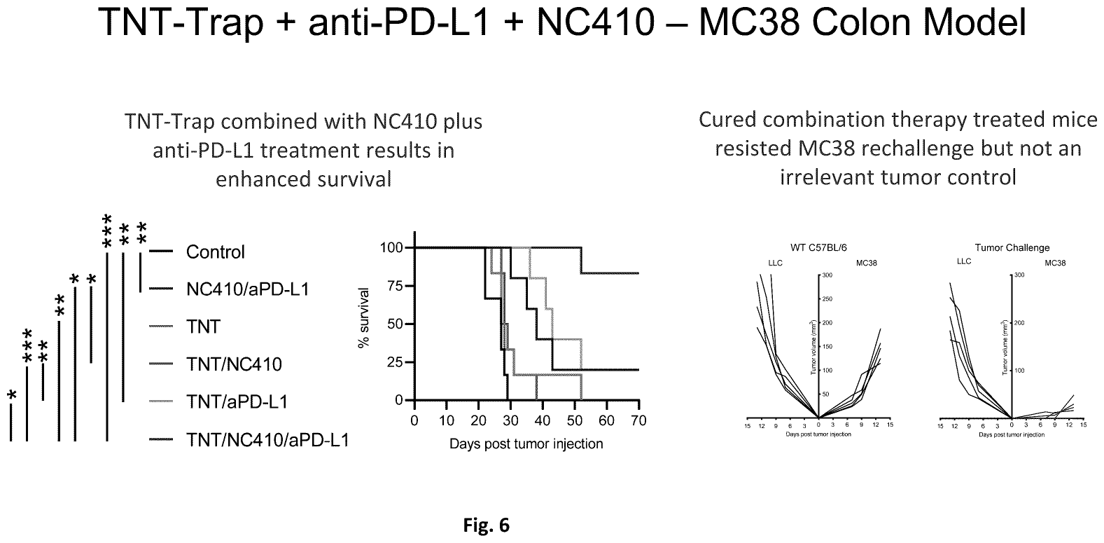

Example 5: TNT-Trap+anti-PD-L1+NC410 (LAIR2 fused to a Fc domain) were used on a MC38 Colon Model. C57BL/6 mice were inoculated s.c. in the flank with 3×10⁵ MC38 cells. Intraperitoneal injections of aPD-L1 (200 μg), LAIR2-Fc (250 μg) LAIR2-Fc, and N-830 (400 μg) were given on days 7, 9, and 11. Tumor volumes were measured every 1 to 3 days. For rechallenge experiment, cured and naive mice were injected with the same MC38 cells or different LLC cells tumor cell line at 12 and 28. Addition of TNT-Trap to the combination NC410 plus anti-PD-L1 resulted in effective tumor control, as can be seen from FIG. 5. As shown in FIG. 6, TNT-Trap combined with NC410 plus anti-PD-L1 treatment resulted in enhanced survival. Furthermore, the cured combination therapy treated mice resisted MC38 rechallenge but not an irrelevant tumor control.

Example 6: Leukocyte-associated immunoglobulin-like receptor (LAIR)-1+ cells strongly adhere to collagen and promote immune evasion by inhibiting NK cell, T cell, B cell, monocyte, and dendritic cell function. LAIR-2 is a secreted protein that can act as a decoy receptor by binding collagen with higher affinity than LAIR-1 and blocks cell membrane LAIR-1 inhibitory signaling. NC410, a dimeric LAIR-2 Fc fusion protein, blocks collagen interaction with LAIR-1. Ramos et al, “Cancer immunotherapy by NC410, a LAIR-2 Fc protein blocking human LAIR-collagen interaction”, Elife. 2021 Jun. 14; 10:e62927. doi: 10.7554/eLife.62927. PMID: 34121658; PMCID: PMC8225389.

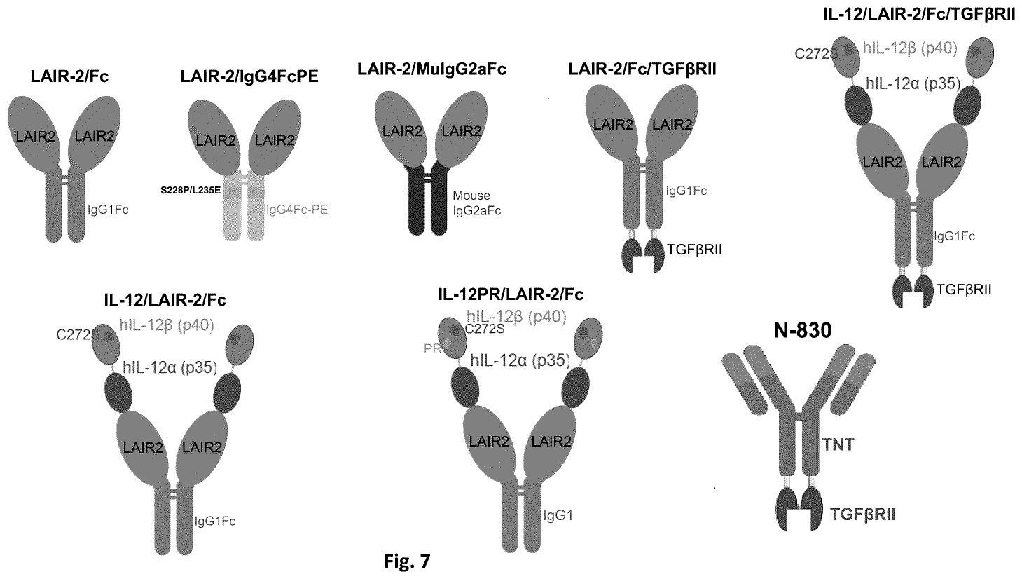

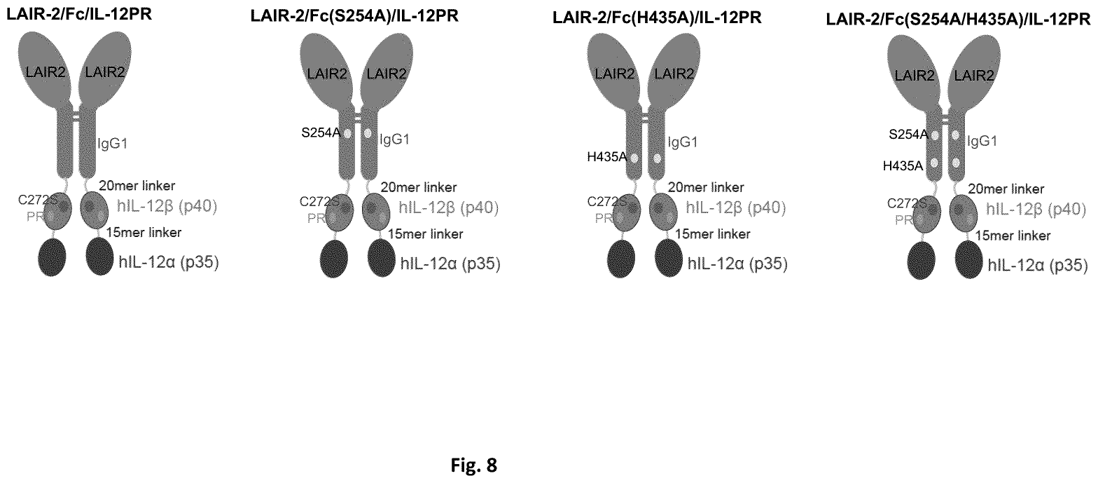

The inventors of this application have developed a variety of LAIR-2 constructs, which are shown in FIGS. 7-10 respectively.

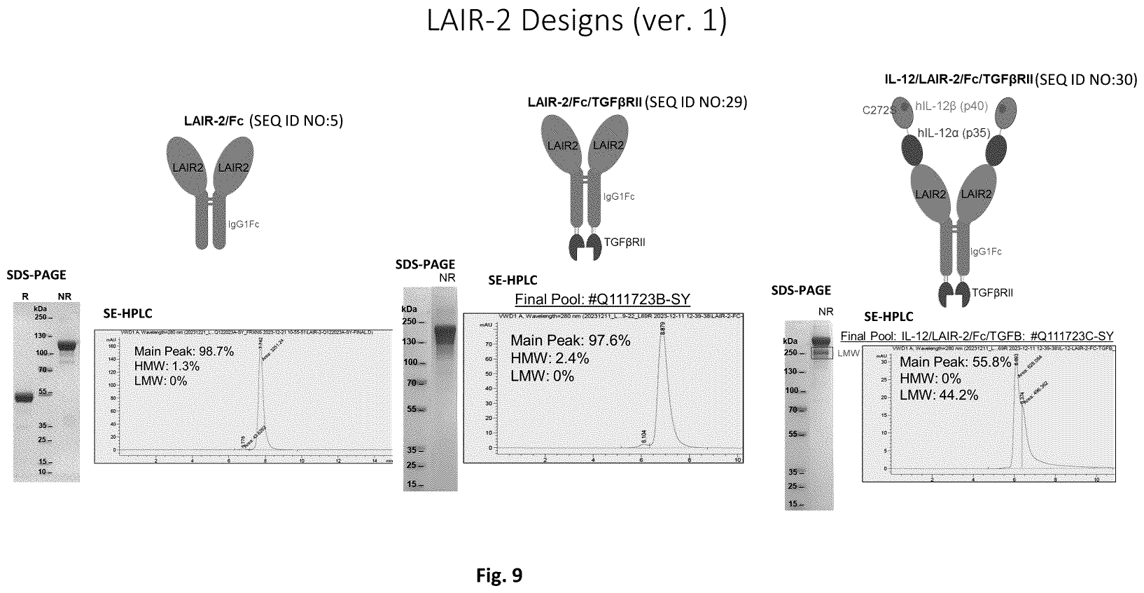

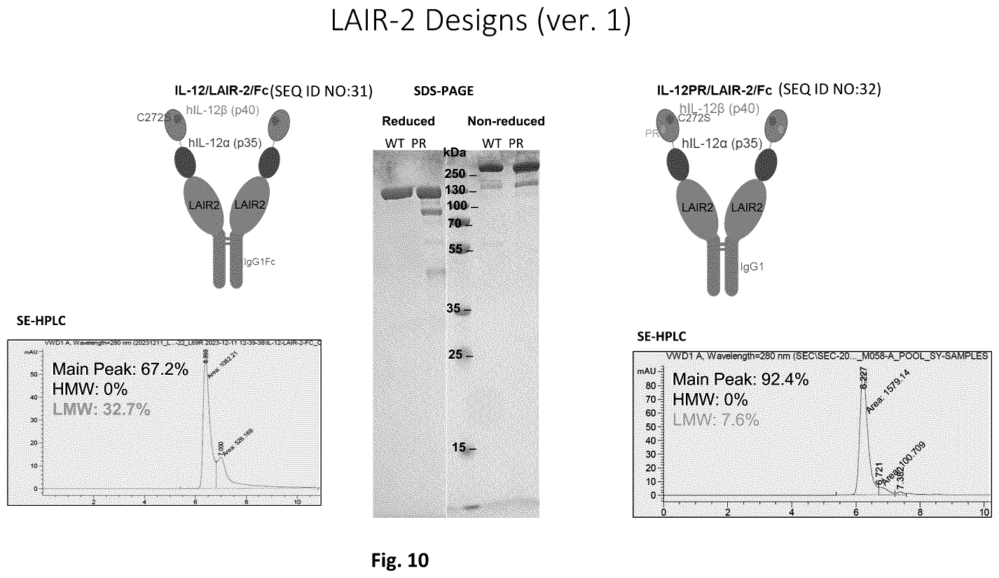

FIGS. 9-10 also show the expression profile of the LAIR-2 constructs, version 1. FIG. 10 shows that the Protease resistant (PR) mutation lowers low molecular weight fragment species. The amino acid sequences of the LAIR-2 constructs of FIGS. 9-10 are shown in SEQ ID NOs 5, 29, 30, 31, and 32 respectively.

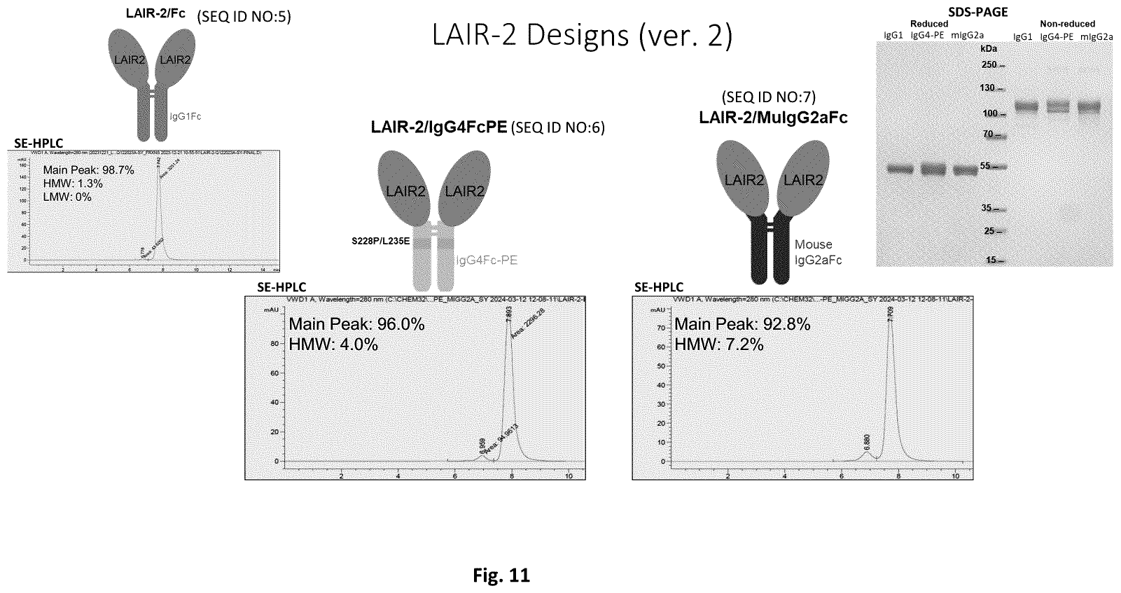

Version 2 of LAIR-2 designs are presented in FIG. 11 and shown in SEQ ID NOs 5, 6, and 7 respectively. The expression profile shows that the LAIR-2 protein is significantly expressed, is soluble, and elutes from the HPLC as a single peak.

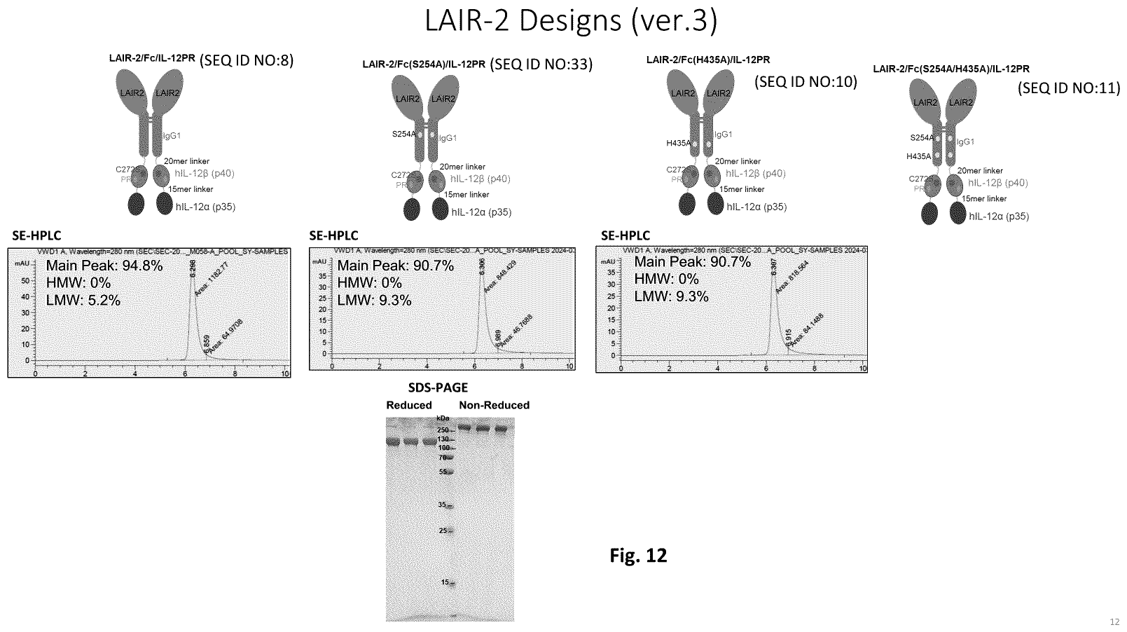

Version 3 of LAIR-2 designs are presented in FIG. 12, and shown in SEQ ID NOs 8, 10, 11, and 33 respectively. The expression profile shows that the LAIR-2 protein is significantly expressed, is soluble, and elutes from the HPLC as a single peak.

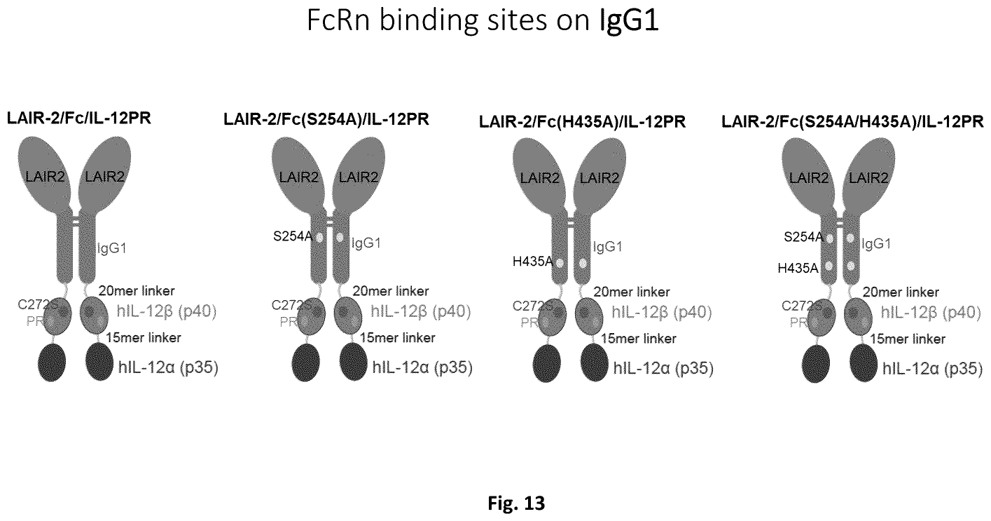

The FcRn binding sites on IgG1 contains I253 and N310 in the CH2 domain and H435 in the CH3 domain. Alanine substitutions in S254 in the CH2 domain and Y436 in the CH3 domain showed reduced binding in vitro to human FcRn. The inventors introduced S254A and H435A Fc mutations into LAIR-2/IL-12 (PR) constructs with the goal of reducing FcRn binding, thereby leading to reduced serum half-life. These constructs are shown in FIG. 13.

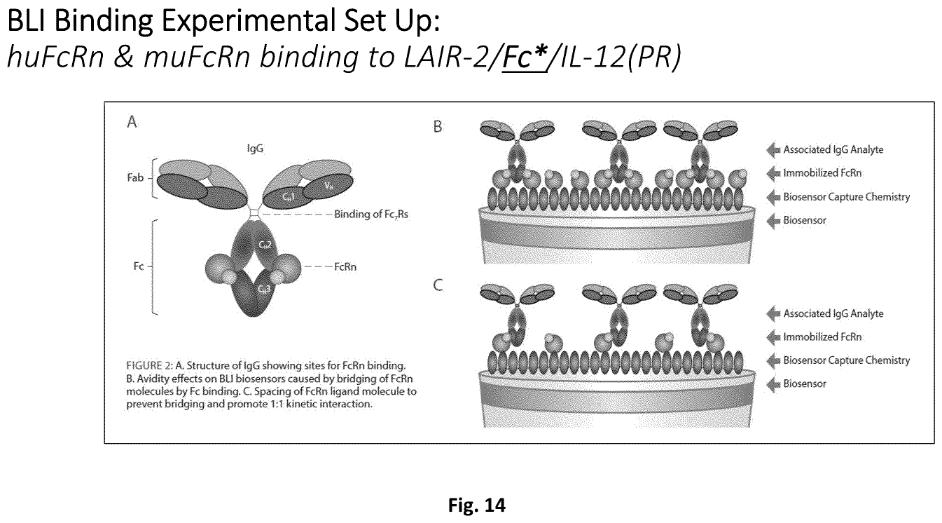

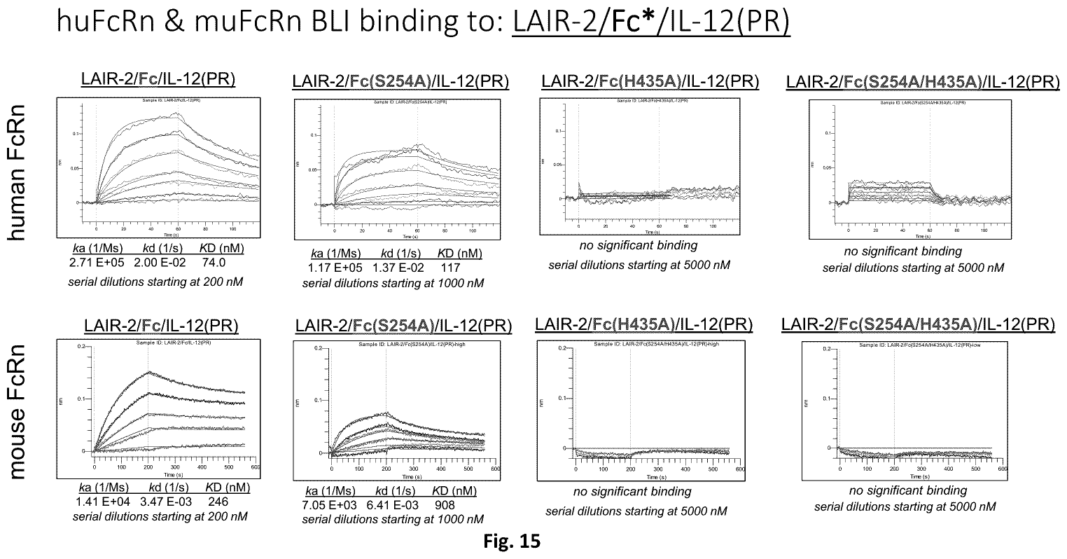

Example 7: The BLI binding experimental set-up to test human FcRn and mouse FcRn binding to LAIR-2/Fc*/IL-12 (PR) is shown in FIGS. 14 and 15. His-tagged FcRn immobilized on HIS1K sensors led to associated IgG analyte. The following constructs were tested:
LAIR-2/Fc/IL-12 (PR)
LAIR-2/Fc (S254A)/IL-12 (PR)
LAIR-2/Fc (H435A)/IL-12 (PR)
LAIR-2/Fc (S254A/H435A)/IL-12 (PR)

LAIR-2/Fc/IL-12 (PR) and LAIR-2/Fc (S254A)/IL-12 (PR) showed significant binding, while the binding of LAIR-2/Fc (H435A)/IL-12 (PR) and LAIR-2/Fc (S254A/H435A)/IL-12 (PR) were negligible.

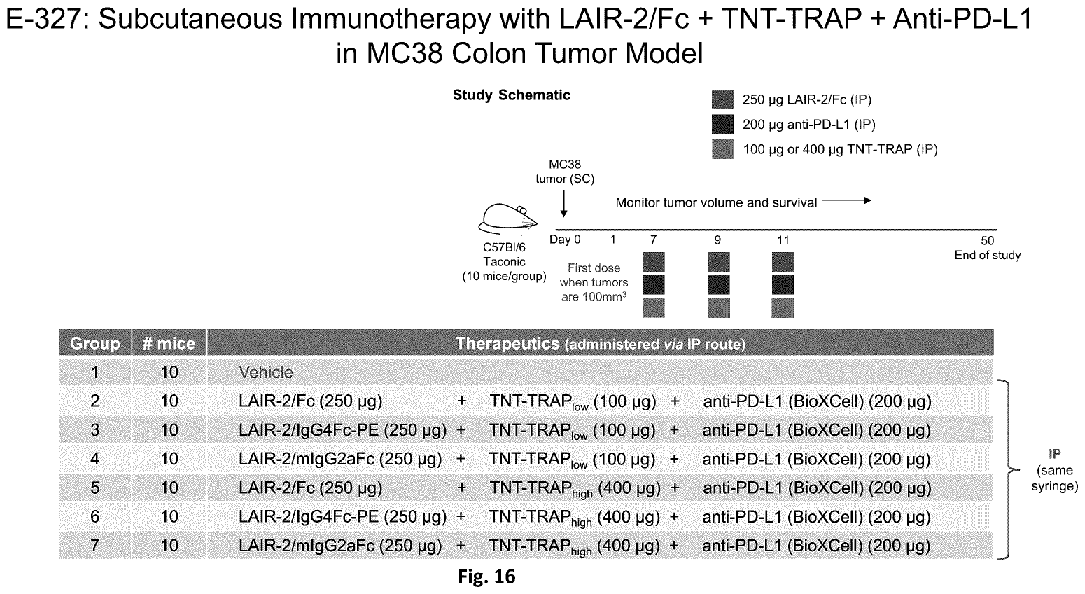

Example 8: Subcutaneous Immunotherapy with LAIR-2/Fc+TNT-TRAP+Anti-PD-L1 in MC38 Colon Tumor Model is shown in FIG. 16. The inventors seek to assess the role of the Fc in LAIR-2/Fc in combination therapy with N-830 and anti-PD-L1. The inventors examine that LAIR2-Fc conditions/prepares the tumor to allow for a reduction in dosing of N-830 (TNT-TGFβ trap). The dosage regime is shown in the table below.

| | # | |
|---|---|---|
| Group | mice | Therapeutics (administered via IP route) |
| 1 | 10 | Vehicle |
| 2 | 10 | LAIR-2/Fc (250 μg) + TNT-TRAP_{low} (100 μg) + anti-PD-L1 (BioXCell) (200 μg) |
| 3 | 10 | LAIR-2/IgG4Fc-PE (250 μg) + TNT-TRAP_{low} (100 μg) + anti-PD-L1 (BioXCell) (200 μg) |
| 4 | 10 | LAIR-2/mIgG2aFc (250 μg) + TNT-TRAP_{low} (100 μg) + anti-PD-L1 (BioXCell) (200 μg) |
| 5 | 10 | LAIR-2/Fc (250 μg) + TNT-TRAP_{high} (400 μg) + anti-PD-L1 (BioXCell) (200 μg) |
| 6 | 10 | LAIR-2/IgG4Fc-PE (250 μg) + TNT-TRAP_{high} (400 μg) + anti-PD-L1 (BioXCell) (200 μg) |
| 7 | 10 | LAIR-2/mIgG2aFc (250 μg) + TNT-TRAP_{high} (400 μg) + anti-PD-L1 (BioXCell) (200 μg) |

Immunotherapeutic evaluation of admixed LAIR-2/Fc+TNT-TGFβ TRAP (N-830)+Anti-PD-L1 intraperitoneal therapy in MC38 mouse colon tumor model is performed. Furthermore, immunotherapeutic assessment of combination therapy of admixed LAIR-2/Fc, TNT-TGFβ TRAP, and anti-PD-L1 administered via intraperitoneal (IP) route on a single MC38 colon tumor challenge in female C57BL/6 mice is performed.

The objective of the study was as follows: Collagens are a primary component of the extracellular matrix and are functional ligands for the inhibitory immune receptor leukocyte-associated immunoglobulin-like receptor (LAIR)-1. LAIR-2 is a secreted protein that can act as a decoy receptor by binding collagen with higher affinity than LAIR-1. Analysis of public human datasets shows that collagens, LAIR-1 and LAIR-2 have unique and overlapping associations with survival in certain tumors. The present inventors have designed and produced novel versions of a dimeric LAIR-2 with a (1) functional IgG1 Fc tail, (2) functional IgG4 Fc tail, and (3) functional mIgG2a Fc tail, as disclosed throughout this disclosure. These new LAIR-2 versions are evaluated in both preclinical and clinical settings.

In a previous study, therapeutic treatment with IP LAIR-2 with a functional IgG1 Fc tail, NC410, in combination with IP TNT-TRAP and anti-PD-L1 resulted in effective MC38 tumor control in C57BL/6 mice, with 5 out of 6 mice clearing tumors after the first challenge. Furthermore, these cured mice were able to prevent new tumor growth upon MC38 rechallenge at 12 weeks post initial implantation.

In this study, the inventors (1) assess the role of the Fc in LAIR-2/Fc in combination therapy with N-830 and anti-PD-L1, and (2) test the hypothesis that LAIR2-Fc conditions the tumor to allow for a reduction in dosing of N-830. To evaluate drug efficacy, naïve female C57BL/6 mice are challenged with MC38 colon tumor cells subcutaneously. Once the tumors have reached 100 mm³ in size (by approximately Day 7-10), the mice will subsequently receive therapeutic drugs, i.e. LAIR-2/Fc or LAIR-2/IgG4Fc-PE, or LAIR-2/mIgG2aFc+N-830+anti-PD-L1 on Days 7, 9, and 11 as outlined in FIG. 16. Treated animals are monitored for well-being 3 days/week, and growth of MC38 tumors will be recorded 2-3 times/week until the end of study on Day 50.

### Reagents:

| | |
|---|---|
| Mouse strain: | C57BL/6NTac |
| Source: | Taconic |
| Age range: | 6-8 weeks |
| Sex: | Female |
| Cell line: | MC38 (murine colon cancer) |
| LAIR-2/Fc: | (IB) |
| LAIR-2/IgG4Fc-PE: | (IB) |
| LAIR-2/mIgG2aFc: | (IB) |
| | TNT-TGFβ TRAP (N-830) |
| Anti-PD-L1: | (BioXcell; Catalog# BE0101 or BP0101) |

### Reagent Quantities Needed:

LAIR-2/Fc: (250 μg/dose)×(3 doses/mouse)×(2 groups of mice)×(10 mice/group)=15 mg

1.2×Overage=18 mg

LAIR-2/IgG4Fc-PE: (250 μg/dose)×(3 doses/mouse)×(2 group of mice)×(10 mice/group)=15 mg

1.2×Overage=18 mg

LAIR-2/mIgG2aFc: (250 μg/dose)×(3 doses/mouse)×(2 groups of mice)×(10 mice/group)=15 mg

1.2×Overage=18 mg

### TNT-TGFβ TRAP (N-830):

(400 μg/dose)×(3 doses/mouse)×(3 group of mice)×(10 mice/group)=36 mg

1.2×Overage=43.2 mg

(100 μg/dose)×(3 doses/mouse)×(3 group of mice)×(10 mice/group)=9 mg

1.2×Overage=10.8 mg

Total=54 mg

Anti-PD-L1: (200 μg/dose)×(3 doses/mouse)×(6 group of mice)×(10 mice/group)=36 mg 1.2×Overage=43.2 mg

MC38 tumor Challenge: The day before tumor challenge, mice are anaesthetized with isoflurane, shaved on the back left flank, and ear tagged. For SC tumor injections, mice are anaesthetized with isoflurane per IACUC protocol. To ensure that there are enough mice with tumors meeting enrollment criteria for immunotherapeutic administration on Day 7-10, 120% of the total number of mice required for this study (84 mice in total) are implanted with 5×10⁵ MC38 tumor cells SC on the left flank in 50 μL sterile PBS on Day 0 of the study.

Immunotherapeutic administration (10 mice/group); For Group 1, 100 μL of sterile PBS per dose will be administered IP on the mouse's left flank, underbelly between hip and nipples. (This volume may need to be adjusted if stock concentrations of purified biologics are too low.) For Groups 2-4:250 μg LAIR-2/Fc or +LAIR-2/IgG4Fc-PE or LAIR-2/mIgG2aFc+100 μg TNT-TGFβ TRAP+200 μg anti-PD-L1 are formulated in 100 μL* volume of sterile PBS per dose and administered IP on the mouse's left flank, underbelly between hip and nipples.

### Example of Group 2 Single Dose/Syringe Formulation:

| | Stock | Volume | Final concentration/ |
|---|---|---|---|
| Therapeutic | concentration | needed | 100 uL dose |
| LAIR-2/Fc | | | 250 μg |
| TNT-TGFβ | | | 100 μg |
| TRAP | | | |
| anti-PD-L1 | | | 200 μg |
| Sterile PBS | — | | — |

### Example of Group 3 Single Dose/Syringe Formulation:

| | Stock | Volume | Final concentration/ |
|---|---|---|---|
| Therapeutic | concentration | needed | 100 μL dose |
| LAIR-2/IgG4Fc-PE | | | 250 μg |
| TNT-TGFβ TRAP | | | 100 μg |
| anti-PD-L1 | | | 200 μg |
| Sterile PBS | — | | — |

### Example of Group 4 Single Dose/Syringe Formulation:

| | Stock | Volume | Final concentration/ |
|---|---|---|---|
| Therapeutic | concentration | needed | 100 μL dose |
| LAIR-2/mIgG2aFc | | | 250 μg |
| TNT-TGFβ TRAP | | | 100 μg |
| anti-PD-L1 | | | 200 μg |
| Sterile PBS | — | | — |

*This volume may need to be adjusted if stock concentrations of purified biologics are too low.

For Groups 5-7, 250 μg LAIR-2/Fc or +LAIR-2/IgG4Fc-PE or LAIR-2/mIgG2aFc+400 μg TNT-TGFβ TRAP+200 μg anti-PD-L1 will be formulated in 100 μL* volume of sterile PBS per dose and administered IP on the mouse's left flank, underbelly between hip and nipples.

### Example of Group 5 Single Dose/Syringe Formulation:

| | Stock | Volume | Final concentration/ |
|---|---|---|---|
| Therapeutic | concentration | needed | 100 μL dose |
| LAIR-2/Fc | | | 250 μg |
| TNT-TGFβ TRAP | | | 400 μg |
| anti-PD-L1 | | | 200 μg |
| Sterile PBS | — | | — |

Example of Group 6 single dose/svringe formulation:

| | Stock | Volume | Final concentration/ |
|---|---|---|---|
| Therapeutic | concentration | needed | 100 μL dose |
| LAIR-2/IgG4Fc-PE | | | 250 μg |
| TNT-TGFβ TRAP | | | 400 μg |
| anti-PD-L1 | | | 200 μg |
| Sterile PBS | — | | — |

Example of Group 7 single dose/syringe formulation:

| | Stock | Volume | Final concentration/ |
|---|---|---|---|
| Therapeutic | concentration | needed | 100 μL dose |
| LAIR-2/mIgG2aFc | | | 250 μg |
| TNT-TGFβ TRAP | | | 400 μg |
| anti-PD-L1 | | | 200 μg |
| Sterile PBS | — | | — |

*This volume may need to be adjusted if stock concentrations of purified biologics are too low.

### Experimental Procedures:

a. Mortality: Daily Observations
b. Clinical Signs Observations: Clinical signs will be observed 3 times a week (usually Monday, Wednesday, and Friday) according to Ullman-Cullere and Foltz, 1999.
c. Tumor Measurements: Measure tumor size 2-3 times/week via digital caliper.
d. Scheduled Euthanasia: Tumor size will be measured every 2-3 days. Mice will be euthanized if/when tumors exceed the 1500 mm³ tumor volume cut-off or upon study termination on Day 50.
e. Unscheduled Euthanasia: Mice will be euthanized for any apparent signs of distress, i.e. if excessive ulceration develops or if pain and/or discomfort is apparent through loss of mobility, failure to groom, guarding of the tumor is observed, or if the animals adopt an abnormal resting posture.
f. In all cases, this study will comply with Omeros SOP 20-000-002, IACUC Policy #6, “Study Endpoints and Health Monitoring Criteria for Studies Involving Tumor Models in Rodents”.

Example 9: The inventors hypothesized that if LAIR2-Fc can be used to disrupt the extracellular collagen matrix (ECM) of tumors and improve tumor infiltration by immune cells, then it would also benefit from additional signals attached via fusion with LAIR2-Fc. These additional signals include:
a. Chemokines: CCL3 (SEQ ID NO:46), CCL4 (SEQ ID NO:47), CCL5 (SEQ ID NO: 48)
b. Growth factors: GM-CSF (SEQ ID NO:34), Flt3L (SEQ ID NO:35)
c. Pro-inflammatory cytokines: IL-12, IL-15
d. Checkpoint Inhibitors: anti-PD-L1, anti-PD-1 (SEQ ID NO:39), anti-CTLA-4 (SEQ ID NO:40)

Alternatively, if LAIR2-Fc can be used to disrupt the ECM of tumors and improve infiltration, it opens the door for low doses of tumor targeted therapies:
a. TNT-IL12 (SEQ ID NO:12, light chain, and SEQ ID NO:13, heavy chain) b. TNT-other fusions as above (TNT-CCL3, etc.)
c. Tumor-specific checkpoint-IL12 (αPD-L1-IL12, αPD-1-IL12, αCTLA-4-IL12, etc)
d. Tumor-specific checkpoint-other fusions as above (αPD-L1-CCL3, etc.)
e. TNT-checkpoint

Tracking LAIR2-Fc in tumors and off-target (tool for dissecting LAIR2 function, not a potential therapeutic) can be done via LAIR2-Fc-Luciferase.

Example 10: In one embodiment, the following sequences are contemplated:

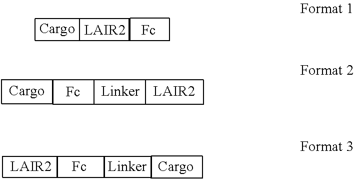

In the above formats, the Cargo may be IL-12, IL-15:IL-15RαSu, GM-CSF (SEQ ID NO: 34), Flt3L (SEQ ID NO:35), CCL19, CCL21 (SEQ ID NO:50), CCL2 (SEQ ID NO:45), CCL3 (SEQ ID NO:46), CCL4 (SEQ ID NO:47), and/or CCL5 (SEQ ID NO:48). In the Formats 1 or 2, preferred cargos include FLT3L, CCL19, CCL21, CCL2, CCL3, CCL4 and CCL5.

In the above sequence formats, the cargo is selected from the group consisting of IL-12 (linked heterodimer, DC-maturation), IL15: IL15RaSu (N-803 or variants with mutations to dampen activity, linked, NK/T-cell activation), GM-CSF (DC maturation), Flt3L (DC maturation), CCL19 (T-cell chemoattractant), CCL21 (T-cell chemoattractant) CCL2 (monocyte, DC, memory T cell Chemoattractant), CCL3 (APC chemoattractant), and CCL5 (T-cell, NK, granulocyte, monocyte, DC chemoattractant). The linker is preferably a (GGGS)ₙ, where n is 2-4, or other linkers that are generally known in the art. The Fc refers to Fc domains with mutations to strengthen/weaken interactions with FcRn and/or effector Fc receptors. The LAIR2 refers to LAIR2 collagen binding domain.

Murine animal studies in syngeneic tumor models are utilized to determine the efficacy of individual and/or combinations of the above candidate therapeutics in the presence or absence of N-803, zabadinostat (Class 1 HDAC inhibitor) and checkpoint blockade antibodies. The effective dose of LAIR2-containing fusion proteins to affect collagen organization is potentially too high for cytokine/chemokine/growth factor fusion proteins to be used as monotherapies at a concentration high enough to disrupt collagen. Therefore, combinations of LAIR2-fusion proteins are explored to maintain high overall LAIR2 concentration, but appropriately low concentrations of cytokine/chemokine/growth factor. Tumor models to be explored include MC38, B16-F10, CT26, etc. Tumors are implanted subcutaneously and LAIR2-fusion proteins will be administered IP or SC when tumors are measurable. Zabadinostat is administered orally.

Example 12: In one embodiment, the following sequences are contemplated:

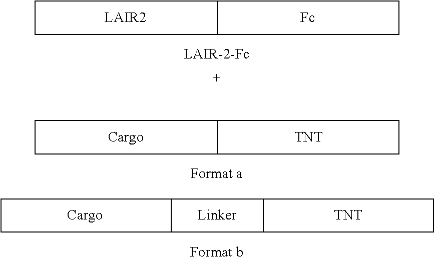

In the above sequence formats, LAIR2 refers to LAIR2 collagen binding domain. The Fc refers to Fc domains with mutations to strengthen/weaken interactions with FcRn and/or effector Fc receptors. The cargo is selected from the group consisting of IL-12 (linked heterodimer, DC-maturation), IL15: IL15RαSu (N-803 or variants with mutations to dampen activity, linked, NK/T-cell activation), GM-CSF (DC maturation), Flt3L (DC maturation), CCL19 (T-cell chemoattractant), CCL21 (T-cell chemoattractant) CCL2 (monocyte, DC, memory T cell Chemoattractant), CCL3 (APC chemoattractant), and CCL5 (T-cell, NK, granulocyte, monocyte, DC chemoattractant). The linker is preferably a (GGGS)ₙ, where n is 2-4, or other linkers that are generally known in the art. The TNT is a tumor-targeting antibody, α-PD-L1, α-double stranded DNA (“TNT”).

Murine animal studies in syngeneic tumor models are utilized to determine the efficacy of LAIR2-Fc in combination with individual or combinations of tumor targeted (TNT) cytokines/chemokines/growth factors. Combinations of the above candidate therapeutics in the presence or absence of N-803, zabadinostat and checkpoint blockade antibodies. Tumor models to be explored include MC38, B16-F10, CT26, etc. Tumors are implanted subcutaneously and LAIR2-fusion proteins and TNT-cargo are administered IP or SC when tumors are measurable. Zabadinostat is administered orally.

Example 13: In one embodiment, the following sequences are contemplated:

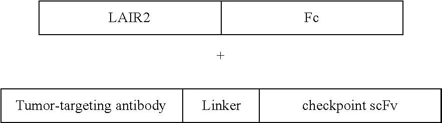

In the above sequence formats, LAIR2 refers to LAIR2 collagen binding domain. The Fc refers to Fc domains with mutations to strengthen/weaken interactions with FcRn and/or effector Fc receptors. The linker is preferably a (GGGS)ₙ, where n is 2-4, or other linkers that are generally known in the art. The TNT is a tumor-targeting antibody, α-PD-L1, α-double stranded DNA (“TNT”). The checkpoint scFv refers to α-PD-L1, α-PD-1, α-CTLA-4, etc.

Murine animal studies in syngeneic tumor models are utilized to determine the efficacy of LAIR2-Fc in combination with individual or combinations of tumor targeted checkpoint inhibiting antibodies. Combinations of the above candidate therapeutics in the presence or absence of N-803 and zabadinostat. Tumor models to be explored include MC38, B16-F10, CT26, etc. Tumors are implanted subcutaneously and LAIR2-fusion proteins and tumor-targeted checkpoint blockade are administered IP or SC when tumors are measurable. Zabadinostat is administered orally.

Example 14: The present application provides amino acid sequences of various polypeptides, proteins, protein complexes, and fusion proteins. An exemplary list of sequences, including LAIR2 designs are shown at the end of the Specification.

The above discussion provides many example embodiments of the inventive subject matter. Although each embodiment represents a single combination of inventive elements, the inventive subject matter is considered to include all possible combinations of the disclosed elements. Thus, if one embodiment comprises elements A, B, and C, and a second embodiment comprises elements B and D, then the inventive subject matter is also considered to include other remaining combinations of A, B, C, or D, even if not explicitly disclosed.

It should be apparent to those skilled in the art that many more modifications besides those already described are possible without departing from the inventive concepts herein. The inventive subject matter, therefore, is not to be restricted except in the spirit of the appended claims. Moreover, in interpreting both the specification and the claims, all terms should be interpreted in the broadest possible manner consistent with the context. In particular, the terms “comprises” and “comprising” should be interpreted as referring to elements, components, or steps in a non-exclusive manner, indicating that the referenced elements, components, or steps may be present, or utilized, or combined with other elements, components, or steps that are not expressly referenced. Where the specification or claims refer to at least one of something selected from the group consisting of A, B, C . . . and N, the text should be interpreted as requiring only one element from the group, not A plus N, or B plus N, etc.

All publications identified herein are incorporated by reference to the same extent as if each individual publication or patent application were specifically and individually indicated to be incorporated by reference. Where a definition or use of a term in an incorporated reference is inconsistent or contrary to the definition of that term provided herein, the definition of that term provided herein applies and the definition of that term in the reference does not apply.

In some embodiments, the numbers expressing quantities of ingredients, properties such as concentration, reaction conditions, and so forth, used to describe and claim certain embodiments of the inventive subject matter are to be understood as being modified in some instances by the term “about.” Accordingly, in some embodiments, the numerical parameters set forth in the written description and attached claims are approximations that can vary depending upon the desired properties sought to be obtained by a particular embodiment. In some embodiments, the numerical parameters should be construed in light of the number of reported significant digits and by applying ordinary rounding techniques. Notwithstanding that the numerical ranges and parameters setting forth the broad scope of some embodiments of the inventive subject matter are approximations, the numerical values set forth in the specific examples are reported as precisely as practicable. The numerical values presented in some embodiments of the inventive subject matter may contain certain errors necessarily resulting from the standard deviation found in their respective testing measurements.

In some embodiments, the numbers expressing quantities of ingredients, properties such as concentration, reaction conditions, and so forth, used to describe and claim certain embodiments of the inventive subject matter are to be understood as being modified in some instances by the term “about.” Accordingly, in some embodiments, the numerical parameters set forth in the written description and attached claims are approximations that can vary depending upon the desired properties sought to be obtained by a particular embodiment. In some embodiments, the numerical parameters should be construed in light of the number of reported significant digits and by applying ordinary rounding techniques. Notwithstanding that the numerical ranges and parameters setting forth the broad scope of some embodiments of the inventive subject matter are approximations, the numerical values set forth in the specific examples are reported as precisely as practicable. The numerical values presented in some embodiments of the inventive subject matter may contain certain errors necessarily resulting from the standard deviation found in their respective testing measurements.

Unless the context dictates the contrary, all ranges set forth herein should be interpreted as being inclusive of their endpoints and open-ended ranges should be interpreted to include only commercially practical values. Similarly, all lists of values should be considered as inclusive of intermediate values unless the context indicates the contrary.

As used in the description herein and throughout the claims that follow, the meaning of “a,” “an,” and “the” includes plural reference unless the context clearly dictates otherwise. Also, as used in the description herein, the meaning of “in” includes “in” and “on” unless the context clearly dictates otherwise.

The recitation of ranges of values herein is merely intended to serve as a shorthand method of referring individually to each separate value falling within the range. Unless otherwise indicated herein, each individual value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided with respect to certain embodiments herein is intended merely to better illuminate the inventive subject matter and does not pose a limitation on the scope of the inventive subject matter otherwise claimed. No language in the specification should be construed as indicating any non-claimed element essential to the practice of the inventive subject matter.

Groupings of alternative elements or embodiments of the inventive subject matter disclosed herein are not to be construed as limitations. Each group member can be referred to and claimed individually or in any combination with other members of the group or other elements found herein. One or more members of a group can be included in, or deleted from, a group for reasons of convenience and/or patentability. When any such inclusion or deletion occurs, the specification is herein deemed to contain the group as modified thus fulfilling the written description of all Markush groups used in the appended claims.

All publications and patent applications herein are incorporated by reference to the same extent as if each individual publication or patent application were specifically and individually indicated to be incorporated by reference. Where a definition or use of a term in an incorporated reference is inconsistent or contrary to the definition of that term provided herein, the definition of that term provided herein applies and the definition of that term in the reference does not apply.

Sequences of the present disclosure

SEQ ID NO: 1
LAIR2-Fc(IgG1)-TGFb Trap(TGFbRII)
From N to C:
Underlined: leader peptide
Italics: LAIR2
Bold: Fc(IgG1)
Underlined: linker
Italics: TGFβRII
MEWSWVFLFFLSVTTGVHSQEGALPRPSISAEPGTVISPGSHVTFMCRGPVGVQTFRLEREDRA
KYKDSYNVFRLGPSESEARFHIDSVSEGNAGLYRCLYYKPPGWSEHSDFLELLVKESSGGPDSP
DTEPGSSAGTVPGTEASGFDAPDKTHTCPPCPAPELLGGPSVFLFPPKPKDTLMISRTPEVTCV
VVDVSHEDPEVKFNWYVDGVEVHNAKTKPREEQYNSTYRVVSVLTVLHQDWLNGKEYKCKVSNK
ALPAPIEKTISKAKGQPREPQVYTLPPSRDELTKNQVSLTCLVKGFYPSDIAVEWESNGQPENN
YKTTPPVLDSDGSFFLYSKLTVDKSRWQQGNVFSCSVMHEALHNHYTQKSLSLSPGKGGGGSGG
GGSGGGGSGGGGSGIPPHVQKSVNNDMIVTDNNGAVKFPQLCKFCDVRFSTCDNQKSCMSNCSI
TSICEKPQEVCVAVWRKNDENITLETVCHDPKLPYHDFILEDAASPKCIMKEKKKPGETFFMCS
CSSDECNDNIIFSEEYNTSNPD
SEQ ID NO: 2
LAIR2-Fc(IgG1)-IL12/LP-single chain IL12-linker-LAIR2(21-152)-
Fc(IgG1)
From N to C:
Underlined: leader peptide
Italics: single chain IL12
Bold: linker
Underlined: LAIR2
Italics: Fc(IgG1)
MEWSWVFLFFLSVTTGVHSIWELKKDVYVVELDWYPDAPGEMVVLTCDTPEEDGITWTLDQSSE
VLGSGKTLTIQVKEFGDAGQYTCHKGGEVLSHSLLLLHKKEDGIWSTDILKDQKEPKNKTFLRC
EAKNYSGRFTCWWLTTISTDLTFSVKSSRGSSDPQGVTCGAATLSAERVRGDNKEYEYSVECQE
DSACPAAEESLPIEVMVDAVHKLKYENYTSSFFIRDIIKPDPPKNLQLKPLKNSRQVEVSWEYP
DTWSTPHSYFSLTFSVQVQGKSKREKKDRVFTDKTSATVICRKNASISVRAQDRYYSSSWSEWA
SVPCSGGGGSGGGGSGGGGSRNLPVATPDPGMFPCLHHSQNLLRAVSNMLQKARQTLEFYPCTS
EEIDHEDITKDKTSTVEACLPLELTKNESCLNSRETSFITNGSCLASRKTSFMMALCLSSIYED
LKMYQVEFKTMNAKLLMDPKRQIFLDQNMLAVIDELMQALNFNSETVPQKSSLEEPDFYKTKIK
LCILLHAFRIRAVTIDRVMSYLNASGGSGGQEGALPRPSISAEPGTVISPGSHVTEMCRGPVGV
QTFRLEREDRAKYKDSYNVFRLGPSESEARFHIDSVSEGNAGLYRCLYYKPPGWSEHSDFLELL
VKESSGGPDSPDTEPGSSAGTVPGTEASGEDAPDKTHTCPPCPAPELLGGPSVFLFPPKPKDTL
MISRTPEVTCVVVDVSHEDPEVKFNWYVDGVEVHNAKTKPREEQYNSTYRVVSVLTVLHQDWLN
GKEYKCKVSNKALPAPIEKTISKAKGQPREPQVYTLPPSRDELTKNQVSLTCLVKGFYPSDIAV
EWESNGQPENNYKTTPPVLDSDGSFFLYSKLTVDKSRWQQGNVFSCSVMHEALHNHYTQKSLSL
SPGK
SEQ ID NO: 3
TNT-TGFβtrap (N830, Light Chain Orf including leader peptide
(LP-Hu51VL-Cκ)
From N to C:
Underlined: leader peptide
Italics: Hu51VL
Bold: Ck
MEWSWVFLFFLSVTTGVHSEIVLTQSPATLSLSPGERATLSCRARQSISNYLHWYQQKPGQAPR
LLIYYASQSISGIPDRFSGSGSGTDFTLTISRLEPEDFAVYYCQQSNSWPLTFGQGTKVEIKRT
VAAPSVFIFPPSDEQLKSGTASVVCLLNNFYPREAKVQWKVDNALQSGNSQESVTEQDSKDSTY
SLSSTLTLSKADYEKHKVYACEVTHQGLSSPVTKSFNRGEC
SEQ ID NO: 4
TNT-TGFβtrap (N830, Heavy Chain Orf including leader peptide
(LP-Hu51VH-CH(IgG1)-linker-TGFβRII
From N to C:
Underlined: leader peptide
Italics: Hu51VH
Bold: CH(IgG1)
Underlined: Linker
Italics: TGFβRII
MEWSWVFLFFLSVTTGVHSQVQLVQSGAEVKKPGASVKVSCKASGYTFTRYWMHWVRQAPGQGL
EWIGAIYPGNSDTSYNQKFKGKATITADTSTNTAYMELSSLRSEDTAVYYCARGEEIGSRRWFA
YWGQGTLVTVSSASTKGPSVFPLAPSSKSTSGGTAALGCLVKDYFPEPVTVSWNSGALTSGVHT
FPAVLQSSGLYSLSSVVTVPSSSLGTQTYICNVNHKPSNTKVDKKVEPKSCDKTHTCPPCPAPE
LLGGPSVFLFPPKPKDTLMISRTPEVTCVVVDVSHEDPEVKFNWYVDGVEVHNAKTKPREEQYN
STYRVVSVLTVLHQDWLNGKEYKCKVSNKALPAPIEKTISKAKGQPREPQVYTLPPSRDELTKN
QVSLTCLVKGFYPSDIAVEWESNGQPENNYKTTPPVLDSDGSFFLYSKLTVDKSRWQQGNVFSC
SVMHEALHNHYTQKSLSLSPGKGGGGSGGGGSGGGGSGGGGSGIPPHVQKSVNNDMIVTDNNGA
VKFPQLCKFCDVRFSTCDNQKSCMSNCSITSICEKPQEVCVAVWRKNDENITLETVCHDPKLPY
HDFILEDAASPKCIMKEKKKPGETFFMCSCSSDECNDNIIFSEEYNTSNPD
SEQ ID NO: 5
LAIR2/IgG1Fc
From N to C:
Underlined: LAIR2
Italics: IgG1Fc
QEGALPRPSISAEPGTVISPGSHVTFMCRGPVGVQTFRLEREDRAKYKDSYNVERLGPSE
SEARFHIDSVSEGNAGLYRCLYYKPPGWSEHSDFLELLVKESSGGPDSPDTEPGSSAGTV
PGTEASGFDAPDKTHTCPPCPAPELLGGPSVFLFPPKPKDTLMISRTPEVTCVVVDVSHE
DPEVKFNWYVDGVEVHNAKTKPREEQYNSTYRVVSVLTVLHQDWLNGKEYKCKVSNKALP
APIEKTISKAKGQPREPQVYTLPPSRDELTKNQVSLTCLVKGFYPSDIAVEWESNGQPEN
NYKTTPPVLDSDGSFFLYSKLTVDKSRWQQGNVFSCSVMHEALHNHYTQKSLSLSPGK
SEQ ID NO: 6
LAIR2/IgG4Fc-PE
From N to C:
Underlined: LAIR2
Italics: IgG4Fc-PE
QEGALPRPSISAEPGTVISPGSHVTFMCRGPVGVQTERLEREDRAKYKDSYNVERLGPSE
SEARFHIDSVSEGNAGLYRCLYYKPPGWSEHSDFLELLVKESSGGPDSPDTEPGSSAGTV
PGTEASGFDAPKYGPPCPPCPAPEFEGGPSVFLFPPKPKDTLMISRTPEVTCVVVDVSQE
DPEVQFNWYVDGVEVHNAKTKPREEQFNSTYRVVSVLTVLHQDWLNGKEYKCKVSNKGLP
SSIEKTISKAKGQPREPQVYTLPPSQEEMTKNQVSLTCLVKGFYPSDIAVEWESNGQPEN
NYKTTPPVLDSDGSFFLYSRLTVDKSRWQEGNVFSCSVMHEALHNHYTQKSLSLSLGK
SEQ ID NO: 7
LAIR2/mIgG2a
From N to C:
Underlined: LAIR2
Italics: mIgG2a
QEGALPRPSISAEPGTVISPGSHVTFMCRGPVGVQTFRLEREDRAKYKDSYNVFRLGPSE
SEARFHIDSVSEGNAGLYRCLYYKPPGWSEHSDFLELLVKESSGGPDSPDTEPGSSAGTV
PGTEASGFDAPEPRGPTIKPCPPCKCPAPNLLGGPSVFIFPPKIKDVLMISLSPIVTCVV
VDVSEDDPDVQISWFVNNVEVHTAQTQTHREDYNSTLRVVSALPIQHQDWMSGKEFKCKV
NNKDLPAPIERTISKPKGSVRAPQVYVLPPPEEEMTKKQVTLTCMVTDFMPEDIYVEWTN
NGKTELNYKNTEPVLDSDGSYFMYSKLRVEKKNWVERNSYSCSVVHEGLHNHHTTKSFSR
TPGK
SEQ ID NO: 8
LAIR2/Fc/IL-12PR (Protease Resistant)
From N to C:
Underlined: LAIR2
Italics: Fc(IgG1)
Bold: Linker
underlined: IL-12PR (Protease Resistant)
QEGALPRPSISAEPGTVISPGSHVTFMCRGPVGVQTFRLEREDRAKYKDSYNVFRLGPSE
SEARFHIDSVSEGNAGLYRCLYYKPPGWSEHSDFLELLVKESSGGPDSPDTEPGSSAGTV
PGTEASGFDAPDKTHTCPPCPAPELLGGPSVFLFPPKPKDTLMISRTPEVTCVVVDVSHE
DPEVKFNWYVDGVEVHNAKTKPREEQYNSTYRVVSVLTVLHQDWLNGKEYKCKVSNKALP
APIEKTISKAKGQPREPQVYTLPPSRDELTKNQVSLTCLVKGFYPSDIAVEWESNGQPEN
NYKTTPPVLDSDGSFFLYSKLTVDKSRWQQGNVFSCSVMHEALHNHYTQKSLSLSPGKGG
GGSGGGGSGGGGSGGGGSIWELKKDVYVVELDWYPDAPGEMVVLTCDTPEEDGITWTLDQ
SSEVLGSGKTLTIQVKEFGDAGQYTCHKGGEVLSHSLLLLHKKEDGIWSTDILKDQKEPK
NKTFLRCEAKNYSGRFTCWWLTTISTDLTFSVKSSRGSSDPQGVTCGAATLSAERVRGDN
KEYEYSVECQEDSACPAAEESLPIEVMVDAVHKLKYENYTSSFFIRDIIKPDPPKNLQLK
PLKNSRQVEVSWEYPDTWSTPHSYFSLTFSVQVQGKDNTEGRVFTDKTSATVICRKNASI
SVRAQDRYYSSSWSEWASVPCSGGGGSGGGGSGGGGSRNLPVATPDPGMFPCLHHSQNLL
RAVSNMLQKARQTLEFYPCTSEEIDHEDITKDKTSTVEACLPLELTKNESCLNSRETSFI
TNGSCLASRKTSEMMALCLSSIYEDLKMYQVEFKTMNAKLLMDPKRQIFLDQNMLAVIDE
LMQALNFNSETVPQKSSLEEPDFYKTKIKLCILLHAFRIRAVTIDRVMSYLNAS
SEQ ID NO: 9
LAIR2/Fc(S254A)/IL-12PR (Weaker FcRN binding. Faster clearance)
Underline: LAIR2
Italics: Fc(S254A)
Bold: linker
Underline: IL-12 PR
QEGALPRPSISAEPGTVISPGSHVTFMCRGPVGVQTFRLEREDRAKYKDSYNVFRLGPSE
SEARFHIDSVSEGNAGLYRCLYYKPPGWSEHSDFLELLVKESSGGPDSPDTEPGSSAGTV
PGTEASGFDAPDKTHTCPPCPAPELLGGPSVFLFPPKPKDTLMIARTPEVTCVVVDVSHE
DPEVKFNWYVDGVEVHNAKTKPREEQYNSTYRVVSVLTVLHQDWLNGKEYKCKVSNKALP
APIEKTISKAKGQPREPQVYTLPPSRDELTKNQVSLTCLVKGFYPSDIAVEWESNGQPEN
NYKTTPPVLDSDGSFFLYSKLTVDKSRWQQGNVFSCSVMHEALHNHYTQKSLSLSPGKGG
GGSGGGGSGGGGSGGGGSIWELKKDVYVVELDWYPDAPGEMVVLTCDTPEEDGITWILDQ
SSEVLGSGKTLTIQVKEFGDAGQYTCHKGGEVLSHSLLLLHKKEDGIWSTDILKDQKEPK
NKTFLRCEAKNYSGRFTCWWLTTISTDLTFSVKSSRGSSDPQGVTCGAATLSAERVRGDN
KEYEYSVECQEDSACPAAEESLPIEVMVDAVHKLKYENYTSSFFIRDIIKPDPPKNLQLK
PLKNSRQVEVSWEYPDTWSTPHSYFSLTFSVQVQGKDNTEGRVFTDKTSATVICRKNASI
SVRAQDRYYSSSWSEWASVPCSGGGGSGGGGSGGGGSRNLPVATPDPGMFPCLHHSQNLL
RAVSNMLQKARQTLEFYPCTSEEIDHEDITKDKTSTVEACLPLELTKNESCLNSRETSFI
TNGSCLASRKTSFMMALCLSSIYEDLKMYQVEFKTMNAKLLMDPKRQIFLDQNMLAVIDE
LMQALNFNSETVPQKSSLEEPDFYKTKIKLCILLHAFRIRAVTIDRVMSYLNAS
SEQ ID NO: 10
LAIR2/Fc(H435A)/IL-12PR (Weaker FcRN binding. Faster clearance)
Underline: LAIR2
Italics: Fc(H435A)
Bold: linker
Underline: IL-12 PR
QEGALPRPSISAEPGTVISPGSHVTFMCRGPVGVQTFRLEREDRAKYKDSYNVERLGPSE
SEARFHIDSVSEGNAGLYRCLYYKPPGWSEHSDFLELLVKESSGGPDSPDTEPGSSAGTV
PGTEASGFDAPDKTHTCPPCPAPELLGGPSVFLFPPKPKDTLMISRTPEVTCVVVDVSHE
DPEVKFNWYVDGVEVHNAKTKPREEQYNSTYRVVSVLTVLHQDWLNGKEYKCKVSNKALP
APIEKTISKAKGQPREPQVYTLPPSRDELTKNQVSLTCLVKGFYPSDIAVEWESNGQPEN
NYKTTPPVLDSDGSFFLYSKLTVDKSRWQQGNVFSCSVMHEALHNAYTQKSLSLSPGKGG
GGSGGGGSGGGGSGGGGSIWELKKDVYVVELDWYPDAPGEMVVLTCDTPEEDGITWTLDQ
SSEVLGSGKTLTIQVKEFGDAGQYTCHKGGEVLSHSLLLLHKKEDGIWSTDILKDQKEPK
NKTFLRCEAKNYSGRFTCWWLTTISTDLTFSVKSSRGSSDPQGVTCGAATLSAERVRGDN
KEYEYSVECQEDSACPAAEESLPIEVMVDAVHKLKYENYTSSFFIRDIIKPDPPKNLQLK
PLKNSRQVEVSWEYPDTWSTPHSYFSLTFSVQVQGKDNTEGRVFTDKTSATVICRKNASI
SVRAQDRYYSSSWSEWASVPCSGGGGSGGGGSGGGGSRNLPVATPDPGMFPCLHHSQNLL
RAVSNMLQKARQTLEFYPCTSEEIDHEDITKDKTSTVEACLPLELTKNESCLNSRETSFI
TNGSCLASRKTSFMMALCLSSIYEDLKMYQVEFKTMNAKLLMDPKRQIFLDQNMLAVIDE
LMQALNFNSETVPQKSSLEEPDFYKTKIKLCILLHAFRIRAVTIDRVMSYLNAS
SEQ ID NO: 11
LAIR2/Fc(S254A/H435A)/IL-12PR (Weaker FcRN binding. Faster
clearance)
Underline: LAIR2
Italics: Fc(S254A/H435A)
Bold: linker
Underline: IL-12 PR
QEGALPRPSISAEPGTVISPGSHVTEMCRGPVGVQTFRLEREDRAKYKDSYNVFRLGPSE
SEARFHIDSVSEGNAGLYRCLYYKPPGWSEHSDFLELLVKESSGGPDSPDTEPGSSAGTV
PGTEASGFDAPDKTHTCPPCPAPELLGGPSVFLFPPKPKDTLMIARTPEVTCVVVDVSHE
DPEVKFNWYVDGVEVHNAKTKPREEQYNSTYRVVSVLTVLHQDWLNGKEYKCKVSNKALP
APIEKTISKAKGQPREPQVYTLPPSRDELTKNQVSLTCLVKGFYPSDIAVEWESNGQPEN
NYKTTPPVLDSDGSFFLYSKLTVDKSRWQQGNVFSCSVMHEALHNAYTQKSLSLSPGKGG
GGSGGGGSGGGGSGGGGSIWELKKDVYVVELDWYPDAPGEMVVLTCDTPEEDGITWTLDQ
SSEVLGSGKTLTIQVKEFGDAGQYTCHKGGEVLSHSLLLLHKKEDGIWSTDILKDQKEPK
NKTFLRCEAKNYSGRFTCWWLTTISTDLTFSVKSSRGSSDPQGVTCGAATLSAERVRGDN
KEYEYSVECQEDSACPAAEESLPIEVMVDAVHKLKYENYTSSFFIRDIIKPDPPKNLQLK
PLKNSRQVEVSWEYPDTWSTPHSYFSLTFSVQVQGKDNTEGRVFTDKTSATVICRKNASI
SVRAQDRYYSSSWSEWASVPCSGGGGSGGGGSGGGGSRNLPVATPDPGMFPCLHHSQNLL
RAVSNMLQKARQTLEFYPCTSEEIDHEDITKDKTSTVEACLPLELTKNESCLNSRETSFI
TNGSCLASRKTSEMMALCLSSIYEDLKMYQVEFKTMNAKLLMDPKRQIFLDQNMLAVIDE
LMQALNFNSETVPQKSSLEEPDFYKTKIKLCILLHAFRIRAVTIDRVMSYLNAS
SEQ ID NO: 12
TNT-IL-12(N812) Light Chain Orf including leader peptide (LP-
Hu51VL-Ck)
From N to C:
Underlined: leader peptide
Italics: Hu51VL
Bold: Ck
MEWSWVFLFFLSVTTGVHSEIVLTQSPATLSLSPGERATLSCRARQSISNYLHWYQQKPGQAPR
LLIYYASQSISGIPDRFSGSGSGTDFTLTISRLEPEDFAVYYCQQSNSWPLTFGQGTKVEIKRT
VAAPSVFIFPPSDEQLKSGTASVVCLLNNFYPREAKVQWKVDNALQSGNSQESVTEQDSKDSTY
SLSSTLTLSKADYEKHKVYACEVTHQGLSSPVTKSFNRGEC
SEQ ID NO: 13
TNT-IL-12(N812) Heavy Chain Orf including leader peptide (LP-
IL12b(p40)(C272S)-linker-IL12a(p35)-Hu51VH-CH(IgG1))
From N to C:
Underlined: leader peptide
Italics: IL-12 PR
Bold: Hu51VH
Underlined: CH(IgG1)
MEWSWVFLFFLSVTTGVHSIWELKKDVYVVELDWYPDAPGEMVVLTCDTPEEDGITWTLDQSSE
VLGSGKTLTIQVKEFGDAGQYTCHKGGEVLSHSLLLLHKKEDGIWSTDILKDQKEPKNKTFLRC
EAKNYSGRFTCWWLTTISTDLTFSVKSSRGSSDPQGVTCGAATLSAERVRGDNKEYEYSVECQE
DSACPAAEESLPIEVMVDAVHKLKYENYTSSFFIRDIIKPDPPKNLQLKPLKNSRQVEVSWEYP
DTWSTPHSYFSLTFSVQVQGKSKREKKDRVFTDKTSATVICRKNASISVRAQDRYYSSSWSEWA
SVPCSGGGGSGGGGSGGGGSRNLPVATPDPGMFPCLHHSQNLLRAVSNMLQKARQTLEFYPCTS
EEIDHEDITKDKTSTVEACLPLELTKNESCLNSRETSFITNGSCLASRKTSFMMALCLSSIYED
LKMYQVEFKTMNAKLLMDPKRQIFLDQNMLAVIDELMQALNENSETVPQKSSLEEPDFYKTKIK
LCILLHAFRIRAVTIDRVMSYLNASLEQVQLVQSGAEVKKPGASVKVSCKASGYTFTRYWMHWV
RQAPGQGLEWIGAIYPGNSDTSYNQKFKGKATITADTSTNTAYMELSSLRSEDTAVYYCARGEE
IGSRRWFAYWGQGTLVTVSSASTKGPSVFPLAPSSKSTSGGTAALGCLVKDYFPEPVTVSWNSG
ALTSGVHTFPAVLQSSGLYSLSSVVTVPSSSLGTQTYICNVNHKPSNTKVDKKVEPKSCDKTHT
CPPCPAPELLGGPSVFLFPPKPKDTLMISRTPEVTCVVVDVSHEDPEVKFNWYVDGVEVHNAKT
KPREEQYNSTYRVVSVLTVLHQDWLNGKEYKCKVSNKALPAPIEKTISKAKGQPREPQVYTLPP
SRDELTKNQVSLTCLVKGFYPSDIAVEWESNGQPENNYKTTPPVLDSDGSFFLYSKLTVDKSRW
QQGNVFSCSVMHEALHNHYTQKSLSLSPGK
SEQ ID NO: 14
Leader peptide
MEWSWVFLFFLSVTTGVHS
SEQ ID NO: 15
LAIR2
QEGALPRPSISAEPGTVISPGSHVTFMCRGPVGVQTFRLEREDRAKYKDSYNVERLGPSESEAR
FHIDSVSEGNAGLYRCLYYKPPGWSEHSDFLELLVKESSGGPDSPDTEPGSSAGTVPGTEASGF
DAP
SEQ ID NO: 16
Fc(IgG1)
DKTHTCPPCPAPELLGGPSVFLFPPKPKDTLMISRTPEVTCVVVDVSHEDPEVKFNWYVDGVEV
HNAKTKPREEQYNSTYRVVSVLTVLHQDWLNGKEYKCKVSNKALPAPIEKTISKAKGQPREPQV
YTLPPSRDELTKNQVSLTCLVKGFYPSDIAVEWESNGQPENNYKTTPPVLDSDGSFFLYSKLTV
DKSRWQQGNVFSCSVMHEALHNHYTQKSLSLSPGK
SEQ ID NO: 17
linker
GGGGSGGGGSGGGGSGGGGS
SEQ ID NO: 18
TGFbRII
GIPPHVQKSVNNDMIVTDNNGAVKFPQLCKFCDVRFSTCDNQKSCMSNCSITSICEKPQEVCVA
VWRKNDENITLETVCHDPKLPYHDFILEDAASPKCIMKEKKKPGETFFMCSCSSDECNDNIIFS
EEYNTSNPD
SEQ ID NO: 19
Single chain IL12: IL12b(p40)(C272S)-linker-IL12a(p35)
IWELKKDVYVVELDWYPDAPGEMVVLTCDTPEEDGITWTLDQSSEVLGSGKTLTIQVKEFGDAG
QYTCHKGGEVLSHSLLLLHKKEDGIWSTDILKDQKEPKNKTFLRCEAKNYSGRFTCWWLTTIST
DLTFSVKSSRGSSDPQGVTCGAATLSAERVRGDNKEYEYSVECQEDSACPAAEESLPIEVMVDA
VHKLKYENYTSSFFIRDIIKPDPPKNLQLKPLKNSRQVEVSWEYPDTWSTPHSYFSLTFSVQVQ
GKSKREKKDRVFTDKTSATVICRKNASISVRAQDRYYSSSWSEWASVPCSGGGGSGGGGSGGGG
SRNLPVATPDPGMFPCLHHSQNLLRAVSNMLQKARQTLEFYPCTSEEIDHEDITKDKTSTVEAC
LPLELTKNESCLNSRETSFITNGSCLASRKTSFMMALCLSSIYEDLKMYQVEFKTMNAKLLMDP
KRQIFLDQNMLAVIDELMQALNENSETVPQKSSLEEPDFYKTKIKLCILLHAFRIRAVTIDRVM
SYLNAS
SEQ ID NO: 20
IL-12b(p40)(C272S)
IWELKKDVYVVELDWYPDAPGEMVVLTCDTPEEDGITWTLDQSSEVLGSGKTLTIQVKEFGDAG
QYTCHKGGEVLSHSLLLLHKKEDGIWSTDILKDQKEPKNKTFLRCEAKNYSGRFTCWWLTTIST
DLTFSVKSSRGSSDPQGVTCGAATLSAERVRGDNKEYEYSVECQEDSACPAAEESLPIEVMVDA
VHKLKYENYTSSFFIRDIIKPDPPKNLQLKPLKNSRQVEVSWEYPDTWSTPHSYFSLTFSVQVQ
GKSKREKKDRVFTDKTSATVICRKNASISVRAQDRYYSSSWSEWASVPCS
SEQ ID NO: 21
linker
GGGGSGGGGSGGGGS
SEQ ID NO: 22
IL12a(p35)
RNLPVATPDPGMFPCLHHSQNLLRAVSNMLQKARQTLEFYPCTSEEIDHEDITKDKTSTVEACL
PLELTKNESCLNSRETSFITNGSCLASRKTSFMMALCLSSIYEDLKMYQVEFKTMNAKLLMDPK
RQIFLDQNMLAVIDELMQALNENSETVPQKSSLEEPDFYKTKIKLCILLHAFRIRAVTIDRVMS
YLNAS
SEQ ID NO: 23
Linker
GGSGG
SEQ ID NO: 24
Hu51VL
EIVLTQSPATLSLSPGERATLSCRARQSISNYLHWYQQKPGQAPRLLIYYASQSISGIPDRFSG
SGSGTDFTLTISRLEPEDFAVYYCQQSNSWPLTFGQGTKVEIK
SEQ ID NO: 25
Ck
RTVAAPSVFIFPPSDEQLKSGTASVVCLLNNFYPREAKVQWKVDNALQSGNSQESVTEQDSKDS
TYSLSSTLTLSKADYEKHKVYACEVTHQGLSSPVTKSFNRGEC
SEQ ID NO: 26
Hu51VH
QVQLVQSGAEVKKPGASVKVSCKASGYTFTRYWMHWVRQAPGQGLEWIGAIYPGNSDTSYNQKF
KGKATITADTSTNTAYMELSSLRSEDTAVYYCARGEEIGSRRWFAYWGQGTLVTVSS
SEQ ID NO: 27
CH(IgG1)
ASTKGPSVFPLAPSSKSTSGGTAALGCLVKDYFPEPVTVSWNSGALTSGVHTFPAVLQSSGLYS
LSSVVTVPSSSLGTQTYICNVNHKPSNTKVDKKVEPKSCDKTHTCPPCPAPELLGGPSVFLFPP
KPKDTLMISRTPEVTCVVVDVSHEDPEVKFNWYVDGVEVHNAKTKPREEQYNSTYRVVSVLTVL
HQDWLNGKEYKCKVSNKALPAPIEKTISKAKGQPREPQVYTLPPSRDELTKNQVSLTCLVKGFY
PSDIAVEWESNGQPENNYKTTPPVLDSDGSFFLYSKLTVDKSRWQQGNVFSCSVMHEALHNHYT
QKSLSLSPGK
SEQ ID NO: 28
TGFbRII
GIPPHVQKSVNNDMIVTDNNGAVKFPQLCKFCDVRFSTCDNQKSCMSNCSITSICEKPQEVCVA
VWRKNDENITLETVCHDPKLPYHDFILEDAASPKCIMKEKKKPGETFFMCSCSSDECNDNIIFS
EEYNTSNPD
SEQ ID NO: 29
IL-12/LAIR2/IgG1Fc
IWELKKDVYVVELDWYPDAPGEMVVLTCDTPEEDGITWTLDQSSEVLGSGKTLTIQVKEFGDAG
QYTCHKGGEVLSHSLLLLHKKEDGIWSTDILKDQKEPKNKTFLRCEAKNYSGRFTCWWLTTIST
DLTFSVKSSRGSSDPQGVTCGAATLSAERVRGDNKEYEYSVECQEDSACPAAEESLPIEVMVDA
VHKLKYENYTSSFFIRDIIKPDPPKNLQLKPLKNSRQVEVSWEYPDTWSTPHSYFSLTFSVQVQ
GKSKREKKDRVFTDKTSATVICRKNASISVRAQDRYYSSSWSEWASVPCSGGGGSGGGGSGGGG
SRNLPVATPDPGMFPCLHHSQNLLRAVSNMLQKARQTLEFYPCTSEEIDHEDITKDKTSTVEAC
LPLELTKNESCLNSRETSFITNGSCLASRKTSFMMALCLSSIYEDLKMYQVEFKTMNAKLLMDP
KRQIFLDQNMLAVIDELMQALNENSETVPQKSSLEEPDFYKTKIKLCILLHAFRIRAVTIDRVM
SYLNASGGSGGQEGALPRPSISAEPGTVISPGSHVTFMCRGPVGVQTFRLEREDRAKYKDSYNV
FRLGPSESEARFHIDSVSEGNAGLYRCLYYKPPGWSEHSDFLELLVKESSGGPDSPDTEPGSSA
GTVPGTEASGFDAPDKTHTCPPCPAPELLGGPSVFLFPPKPKDTLMISRTPEVTCVVVDVSHED
PEVKFNWYVDGVEVHNAKTKPREEQYNSTYRVVSVLTVLHQDWLNGKEYKCKVSNKALPAPIEK
TISKAKGQPREPQVYTLPPSRDELTKNQVSLTCLVKGFYPSDIAVEWESNGQPENNYKTTPPVL
DSDGSFFLYSKLTVDKSRWQQGNVFSCSVMHEALHNHYTQKSLSLSPGK*
SEQ ID NO: 30
IL-12/LAIR2/IgG1Fc
IWELKKDVYVVELDWYPDAPGEMVVLTCDTPEEDGITWTLDQSSEVLGSGKTLTIQVKEFGDAG
QYTCHKGGEVLSHSLLLLHKKEDGIWSTDILKDQKEPKNKTFLRCEAKNYSGRFTCWWLTTIST
DLTFSVKSSRGSSDPQGVTCGAATLSAERVRGDNKEYEYSVECQEDSACPAAEESLPIEVMVDA
VHKLKYENYTSSFFIRDIIKPDPPKNLQLKPLKNSRQVEVSWEYPDTWSTPHSYFSLTFSVQVQ
GKDNTEGRVFTDKTSATVICRKNASISVRAQDRYYSSSWSEWASVPCSGGGGSGGGGSGGGGSR
NLPVATPDPGMFPCLHHSQNLLRAVSNMLQKARQTLEFYPCTSEEIDHEDITKDKTSTVEACLP
LELTKNESCLNSRETSFITNGSCLASRKTSFMMALCLSSIYEDLKMYQVEFKTMNAKLLMDPKR
QIFLDQNMLAVIDELMQALNFNSETVPQKSSLEEPDFYKTKIKLCILLHAFRIRAVTIDRVMSY
LNASGGSGGQEGALPRPSISAEPGTVISPGSHVTEMCRGPVGVQTFRLEREDRAKYKDSYNVER
LGPSESEARFHIDSVSEGNAGLYRCLYYKPPGWSEHSDFLELLVKESSGGPDSPDTEPGSSAGT
VPGTEASGFDAPDKTHTCPPCPAPELLGGPSVFLFPPKPKDTLMISRTPEVTCVVVDVSHEDPE
VKFNWYVDGVEVHNAKTKPREEQYNSTYRVVSVLTVLHQDWLNGKEYKCKVSNKALPAPIEKTI
SKAKGQPREPQVYTLPPSRDELTKNQVSLTCLVKGFYPSDIAVEWESNGQPENNYKTTPPVLDS
DGSFFLYSKLTVDKSRWQQGNVFSCSVMHEALHNHYTQKSLSLSPGK*
SEQ ID NO: 31
LAIR-2/IgG1Fc/TGFβRII
QEGALPRPSISAEPGTVISPGSHVTFMCRGPVGVQTFRLEREDRAKYKDSYNVFRLGPSESEAR
FHIDSVSEGNAGLYRCLYYKPPGWSEHSDFLELLVKESSGGPDSPDTEPGSSAGTVPGTEASGF
DAPDKTHTCPPCPAPELLGGPSVFLFPPKPKDTLMISRTPEVTCVVVDVSHEDPEVKFNWYVDG
VEVHNAKTKPREEQYNSTYRVVSVLTVLHQDWLNGKEYKCKVSNKALPAPIEKTISKAKGQPRE
PQVYTLPPSRDELTKNQVSLTCLVKGFYPSDIAVEWESNGQPENNYKTTPPVLDSDGSFFLYSK
LTVDKSRWQQGNVFSCSVMHEALHNHYTQKSLSLSPGKGGGGSGGGGSGGGGSGGGGSGIPPHV
QKSVNNDMIVTDNNGAVKFPQLCKFCDVRFSTCDNQKSCMSNCSITSICEKPQEVCVAVWRKND
ENITLETVCHDPKLPYHDFILEDAASPKCIMKEKKKPGETFFMCSCSSDECNDNIIFSEEYNTS
NPD
SEQ ID NO: 32
IL12/LAIR2/IgG1Fc/TGFβRII
IWELKKDVYVVELDWYPDAPGEMVVLTCDTPEEDGITWTLDQSSEVLGSGKTLTIQVKEFGDAG
QYTCHKGGEVLSHSLLLLHKKEDGIWSTDILKDQKEPKNKTFLRCEAKNYSGRFTCWWLTTIST
DLTFSVKSSRGSSDPQGVTCGAATLSAERVRGDNKEYEYSVECQEDSACPAAEESLPIEVMVDA
VHKLKYENYTSSFFIRDIIKPDPPKNLQLKPLKNSRQVEVSWEYPDTWSTPHSYFSLTFSVQVQ
GKSKREKKDRVFTDKTSATVICRKNASISVRAQDRYYSSSWSEWASVPCSGGGGSGGGGSGGGG
SRNLPVATPDPGMFPCLHHSQNLLRAVSNMLQKARQTLEFYPCTSEEIDHEDITKDKTSTVEAC
LPLELTKNESCLNSRETSFITNGSCLASRKTSEMMALCLSSIYEDLKMYQVEFKTMNAKLLMDP
KRQIFLDQNMLAVIDELMQALNFNSETVPQKSSLEEPDFYKTKIKLCILLHAFRIRAVTIDRVM
SYLNASGGSGGQEGALPRPSISAEPGTVISPGSHVTEMCRGPVGVQTERLEREDRAKYKDSYNV
FRLGPSESEARFHIDSVSEGNAGLYRCLYYKPPGWSEHSDFLELLVKESSGGPDSPDTEPGSSA
GTVPGTEASGFDAPDKTHTCPPCPAPELLGGPSVFLFPPKPKDTLMISRTPEVTCVVVDVSHED
PEVKFNWYVDGVEVHNAKTKPREEQYNSTYRVVSVLTVLHQDWLNGKEYKCKVSNKALPAPIEK
TISKAKGQPREPQVYTLPPSRDELTKNQVSLTCLVKGFYPSDIAVEWESNGQPENNYKTTPPVL
DSDGSFFLYSKLTVDKSRWQQGNVFSCSVMHEALHNHYTQKSLSLSPGKGGGGSGGGGSGGGGS
GGGGSGIPPHVQKSVNNDMIVTDNNGAVKFPQLCKFCDVRFSTCDNQKSCMSNCSITSICEKPQ
EVCVAVWRKNDENITLETVCHDPKLPYHDFILEDAASPKCIMKEKKKPGETFFMCSCSSDECND
NIIFSEEYNTSNPD
SEQ ID NO: 33
LAIR2/Fc(S254A)/IL-12PR
QEGALPRPSISAEPGTVISPGSHVTFMCRGPVGVQTFRLEREDRAKYKDSYNVERLGPSESEAR
FHIDSVSEGNAGLYRCLYYKPPGWSEHSDFLELLVKESSGGPDSPDTEPGSSAGTVPGTEASGF
DAPDKTHTCPPCPAPELLGGPSVFLFPPKPKDTLMIARTPEVTCVVVDVSHEDPEVKENWYVDG
VEVHNAKTKPREEQYNSTYRVVSVLTVLHQDWLNGKEYKCKVSNKALPAPIEKTISKAKGQPRE
PQVYTLPPSRDELTKNQVSLTCLVKGFYPSDIAVEWESNGQPENNYKTTPPVLDSDGSFFLYSK
LTVDKSRWQQGNVFSCSVMHEALHNHYTQKSLSLSPGKGGGGSGGGGSGGGGSGGGGSIWELKK
DVYVVELDWYPDAPGEMVVLTCDTPEEDGITWTLDQSSEVLGSGKTLTIQVKEFGDAGQYTCHK
GGEVLSHSLLLLHKKEDGIWSTDILKDQKEPKNKTFLRCEAKNYSGRFTCWWLTTISTDLTFSV
KSSRGSSDPQGVTCGAATLSAERVRGDNKEYEYSVECQEDSACPAAEESLPIEVMVDAVHKLKY
ENYTSSFFIRDIIKPDPPKNLQLKPLKNSRQVEVSWEYPDTWSTPHSYFSLTFSVQVQGKDNTE
GRVFTDKTSATVICRKNASISVRAQDRYYSSSWSEWASVPCSGGGGSGGGGSGGGGSRNLPVAT
PDPGMFPCLHHSQNLLRAVSNMLQKARQTLEFYPCTSEEIDHEDITKDKTSTVEACLPLELTKN
ESCLNSRETSFITNGSCLASRKTSFMMALCLSSIYEDLKMYQVEFKTMNAKLLMDPKRQIFLDQ
NMLAVIDELMQALNFNSETVPQKSSLEEPDFYKTKIKLCILLHAFRIRAVTIDRMSYLNAS
SEQ ID NO: 34
GM-CSF
APARSPSPSTQPWEHVNAIQEARRLLNLSRDTAAEMNETVEVISEMFDLQEPTCLQTRLELYKQ
GLRGSLTKLKGPLTMMASHYKQHCPPTPETSCATQIITFESFKENLKDELLVIPFDCWEPVQE
SEQ ID NO: 35
huFLT3L
TQDCSFQHSPISSDFAVKIRELSDYLLQDYPVTVASNLQDEELCGGLWRLVLAQRWMERLKTVA
GSKMQGLLERVNTEIHFVTKCAFQPPPSCLRFVQTNISRLLQETSEQLVALKPWITRQNFSRCL
ELQCQPDSSTLPPPWSPRPLEATAPTAPQ
SEQ ID NO: 36
IgG1-Fc
KYGPPCPPCPAPEFEGGPSVFLFPPKPKDTLMISRTPEVTCVVVDVSQEDPEVQFNWYVDGVEV
HNAKTKPREEQFNSTYRVVSVLTVLHQDWLNGKEYKCKVSNKGLPSSIEKTISKAKGQPREPQV
YTLPPSQEEMTKNQVTLWCLVKGFYPSDIAVEWESNGQPENNYKTTPPVLDSDGSFFLYSRLTV
DKSRWQEGNVFSCSVMHEALHNHYTQKSLSLSLGK
SEQ ID NO: 37
IgG1-Fc-S254A/H435A (decrease FcRn binding)
DKTHTCPPCPAPELLGGPSVFLFPPKPKDTLMIARTPEVTCVVVDVSHEDPEVKENWYVDGVEV
HNAKTKPREEQYNSTYRVVSVLTVLHQDWLNGKEYKCKVSNKALPAPIEKTISKAKGQPREPQV
YTLPPSRDELTKNQVSLTCLVKGFYPSDIAVEWESNGQPENNYKTTPPVLDSDGSFFLYSKLTV
DKSRWQQGNVFSCSVMHEALHNAYTQKSLSLSPGK
SEQ ID NO: 38
IgG1-Fc-YTE (improve FcRn binding)
KTHTCPPCPAPELLGGPSVFLFPPKPKDTLYITREPEVTCVVVDVSHEDPEVKFNWYVDGVEVH
NAKTKPREEQYNSTYRVVSVLTVLHQDWLNGKEYKCKVSNKALPAPIEKTISKAKGQPREPQVY
TLPPSRDELTKNQVSLTCLVKGFYPSDIAVEWESNGQPENNYKTTPPVLDSDGSFFLYSKLTVD
KSRWQQGNVFSCSVMHEALHNHYTQKSLSLSPGK
SEQ ID NO: 39
αPD-1 scFv (N-633-016-D23 scFv)
DIQMTQSPSSLSASVGDRVTITCQASQDISNYLNWYQQKPGKAPKLLIYDASNLETGVPSRFSG
SGSGTDFTFTISSLQPEDIATYYCQQSTAFPFTFGQGTKVEIKGGGGSGGGGSGGGGSGGGGEV
QLVESGGGLVQPGGSLRLSCAASGFTFSSYLMHWVRQAPGKGLEWVSVISLSGGSTYYADSVKG
RFTISRDNSKNTLYLQMNSLRAEDTAVYYCARDRSSWALLGEDVWGQGTLVTVSS
SEQ ID NO: 40
αCTLA-4 scFv (aka αCTLA-4-7 or N-644-007 scFv)
DIQMTQSPSSLSASVGDRVTITCQASQDISNYLNWYQQKPGKAPKLLIYDASNLETGVPSRFSG
SGSGTDFTFTISSLQPEDIATYYCQQGDAWPWTFGQGTKVEIKGGGGSGGGGSGGGGSEVQLVE
SGGGLVQPGGSLRLSCAASGFTFTSYSMHWVRQAPGKGLEWVSGISGSGRSTSYADSVKGRFTI
SRDNSKNTLYLQMNSLRAEDTAVYYCARDLMAFNLVRAGGFDVWGQGTLVTVSS
SEQ ID NO: 41
N-601 αPD-L1 LC
NIQMTQSPSSVSASVGDRVTITCRASQDISRWLAWYQQKPGKAPKLLIYAASSLQSGVPSRFSG
SGSGTDFALTISSLQPEDFATYYCQQADSRFSITFGQGTRLEIKRTVAAPSVFIFPPSDEQLKS
GTASVVCLLNNFYPREAKVQWKVDNALQSGNSQESVTEQDSKDSTYSLSSTLTLSKADYEKHKV
YACEVTHQGLSSPVTKSFNRGEC
SEQ ID NO: 42
N-601 αPD-L1 HC
EVQLVQSGGGLVQPGGSLRLSCAASGFTFSSYSMNWVRQAPGKGLEWVSYISSSSSTIQYADSV
KGRFTISRDNAKNSLYLQMNSLRDEDTAVYYCARGDYYYGMDVWGQGTTVTVSSASTKGPSVFP
LAPSSKSTSGGTAALGCLVKDYFPEPVTVSWNSGALTSGVHTFPAVLQSSGLYSLSSVVTVPSS
SLGTQTYICNVNHKPSNTKVDKKVEPKSCDKTHTCPPCPAPELLGGPSVFLFPPKPKDTLMISR
TPEVTCVVVDVSHEDPEVKFNWYVDGVEVHNAKTKPREEQYNSTYRVVSVLTVLHQDWLNGKEY
KCKVSNKALPAPIEKTISKAKGQPREPQVYTLPPSRDELTKNQVSLTCLVKGFYPSDIAVEWES
NGQPENNYKTTPPVLDSDGSFFLYSKLTVDKSRWQQGNVFSCSVMHEALHNHYTQKSLSLSPGK
SEQ ID NO: 43
TNT LC
EIVLTQSPATLSLSPGERATLSCRARQSISNYLHWYQQKPGQAPRLLIYYASQSISGIPDRFSG
SGSGTDFTLTISRLEPEDFAVYYCQQSNSWPLTFGQGTKVEIKRTVAAPSVFIFPPSDEQLKSG
TASVVCLLNNFYPREAKVQWKVDNALQSGNSQESVTEQDSKDSTYSLSSTLTLSKADYEKHKVY
ACEVTHQGLSSPVTKSFNRGEC
SEQ ID NO: 44
TNT HC
QVQLVQSGAEVKKPGASVKVSCKASGYTFTRYWMHWVRQAPGQGLEWIGAIYPGNSDTSYNQKF
KGKATITADTSTNTAYMELSSLRSEDTAVYYCARGEEIGSRRWFAYWGQGTLVTVSSASTKGPS
VFPLAPSSKSTSGGTAALGCLVKDYFPEPVTVSWNSGALTSGVHTFPAVLQSSGLYSLSSVVTV
PSSSLGTQTYICNVNHKPSNTKVDKKVEPKSCDKTHTCPPCPAPELLGGPSVFLFPPKPKDTLM
ISRTPEVTCVVVDVSHEDPEVKFNWYVDGVEVHNAKTKPREEQYNSTYRVVSVLTVLHQDWLNG
KEYKCKVSNKALPAPIEKTISKAKGQPREPQVYTLPPSRDELTKNQVSLTCLVKGFYPSDIAVE
WESNGQPENNYKTTPPVLDSDGSFFLYSKLTVDKSRWQQGNVFSCSVMHEALHNHYTQKSLSLS
PGK
SEQ ID NO: 45
CCL2
MKVSAALLCLLLIAATFIPQGLAQPDAINAPVTCCYNFTNRKISVQRLASYRRITSSKCPKEAV
IFKTIVAKEICADPKQKWVQDSMDHLDKQTQTPKT
SEQ ID NO: 46
CCL3
MQVSTAALAVLLCTMALCNQFSASLAADTPTACCESYTSRQIPQNFIADYFETSSQCSKPGVIF
LTKRSRQVCADPSEEWVQKYVSDLELSA
SEQ ID NO: 47
CCL4
MKLCVTVLSLLMLVAAFCSPALSAPMGSDPPTACCFSYTARKLPRNFVVDYYETSSLCSQPAVV
FQTKRSKQVCADPSESWVQEYVYDLELN
SEQ ID NO: 48
CCL5
MKVSAAALAVILIATALCAPASASPYSSDTTPCCFAYIARPLPRAHIKEYFYTSGKCSNPAVVF
VTRKNRQVCANPEKKWVREYINSLEMS
SEQ ID NO: 49
CCL19
MALLLALSLLVLWTSPAPTLSGTNDAEDCCLSVTQKPIPGYIVRNFHYLLIKDGCRVPAVVFTT
LRGRQLCAPPDQPWVERIIQRLQRTSAKMKRRSS
SEQ ID NO: 50
CCL21
MAQSLALSLLILVLAFGIPRTQGSDGGAQDCCLKYSQRKIPAKVVRSYRKQEPSLGCSIPAILF
LPRKRSQAELCADPKELWVQQLMQHLDKTPSPQKPAQGCRKDRGASKTGKKGKGSKGCKRTERS
QTPKGP

What is claimed is:

1. A fusion protein comprising Leukocyte-associated immunoglobulin-like receptor 2 (LAIR-2) and a Fc domain.

2. The fusion protein of claim 1, wherein the Fc domain is selected from the group consisting of IgG1Fc, IgG2Fc, and IgG4Fc.

3. The fusion protein of claim 1, further comprising a TGFβ trap, and optionally wherein the TGFβ trap is TGFβRII.

4. The fusion protein of claim 1, further comprising an immunostimulatory domain, wherein the immunostimulatory domain is selected from the group consisting of IL-12, IL-15, IL-15: IL-15RαSu, and N-803.

5. The fusion protein of claim 1, having a polypeptide sequence with at least 80% identity to a sequence selected from the group consisting of SEQ ID NO:1, SEQ ID NO:2, SEQ ID NO: 5, SEQ ID NO:6, SEQ ID NO:7, SEQ ID NO:8, SEQ ID NO:9, SEQ ID NO:10, SEQ ID NO: 11, SEQ ID NO:15, SEQ ID NO:29, SEQ ID NO:30, SEQ ID NO:31, SEQ ID NO: 32, and SEQ ID NO:33.

6. A fusion protein comprising a Tumor Necrosis Targeted (TNT) antibody fused to a TGFβ trap, wherein the TGFβ trap is TGFβRII.

7. The fusion protein of claim 6, having a polypeptide sequence with at least 80% sequence homogeneity to SEQ ID NO:3 or SEQ ID NO:4.

8. A composition comprising:
a. a tumor-targeted antibody fused to a TGFβ trap;
b. LAIR-2 or a fusion protein derivative thereof; and
c. a checkpoint inhibitor antibody or binding domain thereof.

9. The composition of claim 8, wherein the tumor targeted antibody is a TNT antibody or an anti-PD-L1 antibody.

10. The composition of claim 8, wherein the TGFβ trap is a TGFβ antibody.

11. The composition of claim 8, wherein the TGFβ trap is a soluble TGFβ receptor.

12. The composition of claim 11, wherein the tumor-targeted antibody fused to TGFβ trap has a polypeptide sequence with at least 80% sequence homogeneity to SEQ ID NO:3 or SEQ ID NO:4.

13. The composition of claim 8, wherein the LAIR-2 or a fusion protein derivative thereof has a polypeptide sequence with at least 80% identity to a sequence selected from the group consisting of SEQ ID NO:1, SEQ ID NO:2, SEQ ID NO:5, SEQ ID NO:6, SEQ ID NO: 7, SEQ ID NO:8, SEQ ID NO:9, SEQ ID NO: 10, SEQ ID NO:11, SEQ ID NO:15, SEQ ID NO:29, SEQ ID NO:30, SEQ ID NO:31, SEQ ID NO:32, and SEQ ID NO:33.

14. The composition of claim 8, wherein LAIR-2 comprises a stabilized fusion protein derivative.

15. The composition of claim 13, wherein the fusion protein derivative comprises LAIR-2 fused to a tumor targeted antibody, binding domain thereof, or protein comprising a tumor-specific binding domain.

16. The composition of claim 8, wherein LAIR-2 is fused to an immunostimulatory domain, for stimulating T cells and/or Natural Killer (NK) cells.

17. The composition of claim 15, wherein the immunostimulatory domain comprises IL-12, IL-15, IL-15: IL-15RαSu, or N-803.

18. The composition of claim 8, wherein the checkpoint inhibitor antibody comprises a PD-L1 antibody, a PD-1 antibody, or a CTLA-4 antibody.

19. A method of modulating a collagen matrix in a solid tumor, the method comprising contacting the collagen matrix with the composition of claim 8.

20. A method of treating a patient having a solid tumor, the method comprising:
administering to the patient an effective amount of a composition of claim 8.

21. A composition comprising (i) a Tumor Necrosis Targeted (TNT) antibody fused to a TGFβ trap, and (ii) a checkpoint inhibitor.

22. The composition of claim 21, wherein the TGFβ trap is TGFβRII.

23. The composition of claim 21, wherein the TNT antibody fused to a TGFβ trap has a polypeptide sequence with at least 80% sequence homogeneity to SEQ ID NO:3, or SEQ ID NO: 4.

24. The composition of claim 21, wherein the checkpoint inhibitor antibody comprises a PD-L1 antibody, a PD-1 antibody, or a CTLA-4 antibody.

25. The composition of claim 21, wherein the checkpoint inhibitor antibody comprises a PD-L1 antibody.

26. A method of treating a tumor in a patient in need thereof, comprising administering to the patient the composition of claim 21.
